(19) United States
(12) Patent Application Publication
(10) Pub. No.: US 2023/0119606 A1
(43) Pub. Date: Apr. 20, 2023

(54) IMMUNE CELL MODULATORS

(71) Applicant: Octagon Therapeutics, Inc., Cambridge, MA (US)

(72) Inventors: Nathaniel Sherden, Boston, MA (US); Shen Yu, Winchester, MA (US); Isaac Stoner, Somerville, MA (US)

(21) Appl. No.: 17/800,055

(22) PCT Filed: Jan. 29, 2021
(86) PCT No.: PCT/US2021/015846

§ 371 Date: Aug. 16, 2022

Related U.S. Application Data
(60) Provisional application No. 63/004,922, filed on Apr. 3, 2020.
(60) Provisional application No. 62/978,003, filed on Feb. 18, 2020.

(51) Int. Cl. A61K 47/54; A61K 47/64

(52) U.S. Cl. CPC A61K 47/545; A61K 47/64

(57) ABSTRACT

Disclosed are immune cell-selective small molecule compounds that modulate certain immune cell-specific receptors and enzymes, and methods of their synthesis and use to treat proliferative disorders.

### CROSS-REFERENCE TO RELATED APPLICATIONS

This application claims priority to United States provisional application Nos. 62/978,003 filed Feb. 18, 2020, and 63/004,922 filed Apr. 3, 2020, entitled “Immune Cell Modulators”, the contents of each of which are incorporated by reference herein in its entirety.

### FIELD OF THE INVENTION

The present invention is in the fields of medicine and proliferative diseases. More particularly, the invention relates to small molecule, cell-specific inhibitors and modulators of immune cell proteins for the treatment of proliferative disorders including cancer, atherosclerosis, and autoimmune disorders.

### BACKGROUND OF THE INVENTION

“Proliferative diseases” (PDs) is a unified term to describe several disease types where excessive proliferation of cells and tumor of cellular matrix contribute significantly to the pathogenesis. PDs encompass cancer, inflammatory diseases such as atherosclerosis (AS), and many autoimmune diseases such as rheumatoid arthritis (RA), inflammatory bowel diseases (IBD), psoriasis as well as infectious diseases of bacterial, viral or fungal nature. As such, PDs affects a vast number of populations. For example, autoimmune disease affects 3% to 5% of the population. There are more than 100 distinct autoimmune disorders; the majority involve multiple genetic and environmental factors. The concordance of autoimmune disease in identical twins is 12% to 67%, highlighting the complexity of underlying disease mechanisms and the potential importance of stochastic or epigenetic phenomena.

The first generation therapies for PDs that demonstrated clinical utility were antiproliferative agents such as cancer chemotherapy agents and immunosuppressants. These compounds non-selectively affect rapidly proliferating cells, usually by interfering with cellular metabolism, and thereby exhibiting a range of toxicities including bone marrow suppression, GI-toxicity (affecting intestine epithelial cells or crypt cells), and hair loss (affecting hair follicles). Rapidly proliferating lymphocytes are involved in normal immune function and are critical in controlling infection and in cancer surveillance. Therefore, non-selective suppression of lymphocytes also exposes patients to elevated risk of opportunistic infections as well as neoplasia.

Biologic therapies have also been developed that are based on highly selective inhibitors including monoclonal antibodies, that modify specific inflammatory or effector pathways active in autoimmune disorders (e.g., TNF inhibitors, IL-6 inhibitor, IL-12 inhibitor, BLyS inhibitor CTLA4-IgGs and anti-CD20 antibodies).

Unfortunately, many of these biologics have significant drawbacks. For example, the anti-CD20 antibody, Rituximab (Rituxan), widely used in B-cell malignancy treatment as well as autoimmune diseases, triggers cell death via antibody-dependent cellular cytotoxicity (ADCC) when it binds to CD20 on a B-cell surface. However. Rituximab is known to cause headache and back pain, in addition to possessing a slow administration infusion rate (50 mg/hr), which can take up to eight hours. In addition, its administration increases the risk of infections and malignancies as it depletes B-cells; normal B-cell functions are essentially absent for patients treated with Rituximab. Treatments like Intravenous Immune Globulin (IVIG) can partially restore B-cell functions, but this method also has severe toxicity liabilities.

In autoimmune disorders like RA, anti-TNF biologic drugs such as Adalimumab (Humira, also used in other autoimmune disorders such as IBDs and psoriasis) are often used as front-line treatment. Unfortunately, anti-TNF biologics generally suffer from low response rate, elevated risk for tumors and infections and pain at injection site. For these reasons, it is often necessary to combine biologic drugs with other small molecule chemotherapeutic agents or disease-modifying antirheumatic drugs (DMARDs) to achieve satisfactory therapeutic effects. Examples of such combination therapies include the combination of Rituximab with four chemotherapeutic agents (known as the R-CHOP regimen, Rituximab with cyclophosphamide, hydroxydaunorubicin, oncovin, prednisone/prednisolone) in the treatment of non-Hodgkin lymphomas, and the combination of Adalimumab with methotrexate in treatment of RA. However, when combining with these non-selective small molecule drugs, the advantage of cell-selectivity of the antibodies is lost.

Thus, improved antiproliferative drugs that are disease-modifying and immune cell-type-selective are highly desired.

### SUMMARY OF THE INVENTION

It has been discovered that certain enzymes and receptors are highly expressed in specific immune cell populations. It has also been discovered that certain small molecule compounds that inhibit and/or modulate these receptors or enzymes can be chemically optimized for guidance to and selection for certain immune cell types, and that these compounds exhibit a high level of immunomodulatory and anti-proliferative effects in cell-based assays.

This discovery has been exploited to develop the present invention, which, in part, is directed to immune cell-selective inhibitor or modulator compounds (“CSIMs” or “CSIM compounds”), and to methods of their synthesis and use to treat proliferative disorders of immune cells such as certain cancers, atherosclerosis and autoimmune disorders.

In one aspect, the disclosure provides a CSIM compound having immunomodulatory or anti-proliferative properties, comprising: a bait comprising a molecule which targets the CSIM to an immune cell; and a payload which is a small molecule modulator of an enzyme in, or a receptor on, the immune cell.

In some embodiments, the payload is an inhibitor of a glucocorticoid receptor.

In particular embodiments, the payload is Prednisone, Budesonide, Prednisolone, Dexamethasone, or Hydrocortisone.

In other embodiments, the payload is an inhibitor of a Janus kinase. In certain embodiments the payload is Tofacitinib (Xeljanz), Baricitinib, Oclacitinib. Ruxolitinib, Filgotinib, Decernotinib, Upadacitinib, or Peficitinib.

In yet other embodiments, the payload is an inhibitor of calcineurin. In particular embodiments, the payload is Cyclosporine, Voclosporin, Tacrolimus, or Pimecrolimus.

In other embodiments, the payload is an inhibitor of mammalian target of rapamycin (mTOR). In certain embodiments, the payload is Rapamycin or Everolimus.

In yet other embodiments, the payload is an inhibitor of dihydrofolate reductase (DHFR).

In another embodiment, the payload is an inhibitor of an enzyme involved in purine synthesis and metabolism. In certain embodiments, the payload is an inhibitor of adenylosuccinate synthase, adenylosuccinate lyase, inosine-monophosphate dehydrogenase, GMP synthase, or glutamine-phosphoribosyl pyrophosphate amidotransferase (GPAT).

In still other embodiments, the payload is a folic acid analog. In one embodiment, the payload is methotrexate.

In yet other embodiments, the payload is a purine analog. In particular embodiments, the payload is azathioprine or mercaptopurine.

In some embodiments, the payload is an inhibitor of dihydroorotate dehydrogenase (DHODH). In certain embodiments, the payload is leflunomide or brequinar.

In other embodiments, the payload is a modulator of the sphingosine-1-phosphate receptor (S1P) receptor. In certain embodiments, the payload is Fingolimod, Ozanimod, or Ponesimod.

In another aspect, the disclosure provides a pharmaceutical formulation comprising a formulation comprising a first CSIM compound or a pharmaceutically acceptable salt thereof, and a pharmaceutically acceptable carrier.

In some embodiments, the formulation further comprises a second CSIM compound different than the CSIM compound.

In some embodiments, the formulation further comprises an immunomodulatory or anti-proliferative compound that is not the CSIM compound.

In yet another aspect, the disclosure provides a method of treating a proliferative disorder in a subject, comprising administering a therapeutically effective amount of a pharmaceutical formulation comprising a CSIM compound to the subject. In some embodiments, the pharmaceutical formulation administered comprises at least two different CSIM compounds. In other embodiments, the pharmaceutical formulation administered comprises an immunomodulatory or anti-proliferative compound. In another embodiment, the method further comprises administering a second pharmaceutical formulation comprises a CSIM compound different than the CSIM compound in the first pharmaceutical formulation.

In another aspect, the disclosure provides a method of treating a proliferative disorder in a subject, comprising administering to the subject an amount of the pharmaceutical formulation according to the disclosure effective to inhibit or reduce a symptom of the proliferative disorder.

In still another aspect, the disclosure provides a method of inhibiting the ability of an immune cell to proliferate, comprising contacting the cell with an amount of any one of the CSIMs according to the disclosure effective to inhibit the cell's ability to proliferate.

The disclosure also provides a method of synthesizing a CSIM compound according to the disclosure, comprising the protocols set forth in FIGS. 2-4.

In another aspect, the disclosure provides a method of synthesizing a CSIM compound having structure:

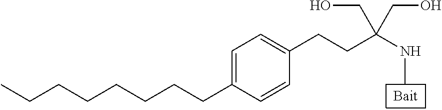 [SMILES: CCCCCCCCc1ccc(CCC(CO)(CO)NC)cc1]

comprising the protocol set forth in FIG. 4.

### DESCRIPTION OF THE DRAWING

The foregoing and other objects of the present disclosure, the various features thereof, as well as the disclosure itself may be more fully understood from the following description, when read together with the accompanying drawings in which:

FIG. 1 is a diagrammatic representation of an acquisition protocol for a representative Payload, wherein all boxed entities represent chemical mater, either a whole molecule, or where joined by a line, represent a portion of a molecule connected by one or more chemical bonds to the other portion(s) as indicated by the connecting line; arrows indicate actions such as synthetic step(s); solid lines for both boxes and arrows represent required steps and molecules, or portions thereof; and dashed lines for boxes and arrows represent steps and molecules or portions thereof that may be conditional;

FIG. 2 is a diagrammatic representation of a synthesis protocol for a representative Bait, wherein: all boxed entities represent chemical mater—either a whole molecule or where joined by a line represent a portion of a molecule connected by one or more chemical bonds to the other portion(s) as indicated by the connecting line; arrows indicate actions such as synthetic step(s), solid lines for both boxes and arrows represent required steps and molecules or portions thereof, and dashed lines for boxes and arrows represent steps and molecules or portions thereof that may be conditional;

FIG. 34 is a diagrammatic representation of a final assembly protocol for a representative CSIM according to the disclosure, wherein all boxed entities represent chemical mater—either a whole molecule or where joined by a line represent a portion of a molecule connected by one or more chemical bonds to the other portion(s) as indicated by the connecting line; arrows indicate actions such as synthetic step(s), solid lines for both boxes and arrows represent required steps and molecules or portions thereof, and dashed lines for boxes and arrows represent steps and molecules or portions thereof that may be conditional; and

FIG. 4 is a diagrammatic representation of a synthetic protocol for a CSIM of the present disclosure.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “modulates” or “modulator” refers to the ability of a CSIM to change (e.g., to inhibit, to stop, to reduce, to block, or to increase)) the activity of a protein such as an enzyme in, or receptor on, the immune cell to which it is directed or targeted, resulting in a change in the physiology of the immune cell, including but not limited to, their proliferation rates, production of cytokines (e.g., interleukins, TNFs, interferons), chemokines, immunoglobulins, or other secreted factors, or immune response towards pathogens such as phagocytosis. The changes may also be the response of the targeted immune cells to stimulus, for example, by mitogens, antigens, secreted factors from other immune and non-immune cells, or direct interaction with other immune and non-immune cells.

The term “treat,” “treated,” “treating,” or “treatment” includes the diminishment or alleviation of at least one symptom associated or caused by the state, disorder or disease being treated. In certain embodiments, the treatment comprises bringing into contact with an infection an effective amount of an anti-infective formulation of the disclosure for conditions related to infections.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, but are not limited to, livestock and pets, such as ovine, bovine, porcine, canine, feline and marine mammals.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “proliferative disorders” refers to excessive, abnormal proliferation or growth of cells and turnover of cellular matrix contribute significantly to the pathogenesis of diseases, including, but not limited to, cancer, tumor, hyperplasias, angiogenesis, atherosclerosis, smooth muscle proliferation in the blood vessels (e.g. stenosis or restenosis following angioplasty), fibrosis (e.g. idiopathic pulmonary fibrosis, renal fibrosis), autoimmune lymphoproliferative syndrome (ALPS) and cirrhosis of the liver. Because most autoimmune diseases involve clonal expansion of lymphocytes, the term “proliferative disorders” also encompasses autoimmune disorders including, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN). Balo disease, Behcet's disease, Benign mucosal pemphigoid. Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block. Coxsackie myocarditis, CREST syndrome, Crohn's disease, Dermatitis herpetiformis, Dermatomyositis. Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis. Narcolepsy, Neonatal Lupus. Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis). Parsonage-Turner syndrome, Pemphigus, Peripheral neuropathy. Perivenous encephalomyelitis, Pernicious anemia (PA), POEMS syndrome, Polyarteritis nodosa, Polyglandular syndromes type I, II, III, Polymyalgia rheumatica. Polymyositis, Postmyocardial infarction syndrome. Postpericardiotomy syndrome. Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis. Reflex sympathetic dystrophy, Relapsing polychondritis. Restless legs syndrome (RLS), Retroperitoneal fibrosis. Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease.

The terms “affected cell” and “affected proliferating cell” encompasses a cell affected by a proliferation disorder which is therefore proliferating abnormally or normally proliferating in response to an abnormal signal, such as, but not limited to, cancer, tumor, autoimmune, and dermatologic disorders such psoriasis, eczema, and ichthyosis and other abnormally proliferating cells such as in autoimmune disorders.

As used herein, the term “pharmaceutically acceptable” refers to a material, such as a carrier or diluent, which does not abrogate the biological activity or properties of the CSIM compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition” refers to the CSIM.

The term “pharmaceutical composition”, or “formulation” refers to at least one of the same or different CSIM or/and salt thereof, according to the disclosure in a pharmaceutically acceptable carrier, and may encompasses other components such as at least one immunomodulatory or antiproliferative compound that is not a CSIM according to the disclosure.

An “oral dosage form” includes a unit dosage form prescribed or intended for oral administration.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. In an embodiment, C₁-C₆ alkyl groups are provided herein. Nonlimiting examples include methyl, ethyl, propyl, isopropyl, butyl, isobutyl, tert butyl, pentyl, neopentyl, and hexyl. Other nonlimiting examples of C₁-C₆ alkyl include ethyl, methyl, isopropyl, isobutyl, n-pentyl, and n-hexyl.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cn n alkenyl” refers to an alkenyl group having n to m carbons. For example, the alkenyl moiety contains 2 to 6, 2 to 4, or 2 to 3 carbon atoms. Exemplary alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “allyl” employed alone or in combination with other terms, refers to an sp³ carbon atom adjacent to an alkenyl group as defined above.

As used herein, the term “halo” or “halogen” alone or as part of another substituent means, unless otherwise stated, a fluorine, chlorine, bromine, or iodine atom.

As used herein, the term “aromatic” refers to a carbocycle or heterocycle with one or more polyunsaturated rings and having aromatic character, i.e., having (4n+2) delocalized n elections where n is an integer).

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. Aryl groups may have 6 carbon atoms, six to ten carbon atoms, or six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclo-penta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]-triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl and heteroaryl moiety may be bonded or otherwise attached to a designated moiety through differing ring atoms (i.e., shown or described without denotation of a specific point of attachment), then all possible points am intended, whether through a carbon atom or, for example, a trivalent nitrogen atom. For example, the term “pyridinyl” means 2-, 3- or 4-pyridinyl, and the term “thienyl” means 2- or 3-thioenyl.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The present disclosure provides small molecule inhibitor or modulator compounds (CSIMs) that are proliferative disease-modifying and immunomodulatory. These CSIMs target and inhibit or modulate certain proteins such as enzymes found in immune cells or receptors found on immune cells.

### CSIM Targets

Exemplary immune cell types to which CSIMs of the disclosure are targeted include, but are not limited to, those in Table 1 below.

TABLE 1

| Immune Cells Populations | Immune Phenotype |
|---|---|
| B cell | CD19⁺ |
| “Helper” T cells | CD4⁺CD8⁻ |
| “Effector” T cells | CD8⁺CD4⁻ |
| Natural Killer (NK) Cells | CD16⁺CD56⁺ |
| Mast Cells | CD23⁺CD117⁺ |
| Neutrophils | CD10⁺ CD13⁺ CD15⁺ CD33+ CD141⁺ CD178⁺ |
| Basophils | CD9⁺ CD23⁺ CD24⁺ CD32⁺ CD43⁺ CD69⁺ |
| Eosinophils | CD9⁺ CD23⁺ CD24⁺ CD32⁺ CD43⁺ CD49d⁺ |
| Monocytes | CD14⁺ CD16⁺ CD64⁺ CD31⁺ CD62L⁺ CD115⁺ CD192⁺ |
| Macrophages | CD14⁺ CD16⁺ CD64⁺ CD68⁺ CD71⁺ CCR5⁺ |
| B Cell Subset | Immune Phenotype |
| Transitional | CD20⁺ CD27⁻ CD38^{hi} IgM⁺ CD24^{hi} BR3⁺ |
| Follicular | IgM^{lo} CD23⁺ CD93⁻ CD19⁺ CD20⁺ CD21⁺ CD22⁺ |
| Marginal zone | IgM^{hi} IgD^{lo} CD1c⁺ CD24⁺ CD19⁺ CD20⁺ CD21⁺ |
| Germinal center | CD20⁺ CD38⁺ BR3⁺ IgD⁻ |
| Plasma cells | CD20⁻ CD38^{hi} CD27^{hi} CD138⁺ TACI⁺ and/or |
| | BCMA⁺ CD126⁺ CD319⁺ CD78⁺ |
| Memory B Cell | CD20⁺ CD38⁻ CD27⁺ CD80⁺ CD84⁺ CD86⁺ |
| T Cell Subset | Immune Phenotype |
| CD8 Memory T cell | CD3⁺ CD8⁺ CD45RO⁺ |
| CD8 cytotoxic T cell | CD3⁺ CD8⁺ Granzyme A/B⁺ |
| Treg | CD25⁺ CD39⁺ CD73⁺ CD103⁺ |
| CD4 memory T Cell | CD3⁺ CD4⁺ CD25⁺ CD45RO⁺ CD62L⁺ |
| Th1 | CD178⁺ CD183⁺ CD195⁺ CD212⁺ CD218a⁺ |
| Th2 | CD183⁺ CD193⁺ CD194⁺ CD198⁺ CD294⁺ |
| Th9 | CD196⁺ IL-9⁺ |
| Th17 | CD45RO⁺ CD126⁺ CD161⁺ CD194⁺ CD196⁺ IL-13α1⁺ |
| | IL-21R⁺ |
| Th22 | CCR10⁺ CD140a⁺ CD140b⁺ CD194⁺ CD196⁺ |
| Tfh | CD185⁺ CD279⁺ |
| Macrophage Subset | Immune Phenotype |
| M1 | CD68⁺ CD86⁺ CD282⁺ CD284⁺ |
| M2 | CD163⁺ CD200R⁺ CD206⁺ |
| TAM | CD81⁺ CD106⁺ Dectin-1⁺ VEGF⁺ |
| CD169⁺ macrophage | CD106⁺ CD169⁺ CD206⁺ |
| TCR⁺ macrophage | CD3⁺ ZAP70⁺ TCRa/β⁺ |

CSIMs are targeted to a particular immune cell type via a moiety that serves as a substrate for one or more enzymes present only in the targeted cell, or which specifically binds to a receptor present on the targeted cells Cell type specificity can be conferred in a variety of ways. Differential permeability, differential activation of a prodrug, differential degradation, and differential enzymatic activation, and/or differential expulsion from different cell types are nonlimiting representative ways in which a drug can be activated preferentially in one cell type compared to others. For example, CSIMs according to the disclosure may be optimized to achieve immune cell-selective accumulation based on interaction with a receptor or enzymes that are highly expressed on or in in the particular immune cell.

One receptor targeted by CSIMS according to the disclosure is the glucocorticoid receptor. This receptor binds corticosteroids, and which when activated as a glucocorticoid receptor-glucocorticoid complex, up-regulates the expression of anti-inflammatory proteins in the nucleus and represses the expression of proinflammatory proteins in the cytosol such as IL-1, IL-2, IL-3, IL-4, IL-5, IL-6, IL-8 and IFN-γ.

Another receptor targeted by CSIMs according to the disclosure is the sphingosine-1-phosphate (SIP) receptor, which, when bound by certain agonist molecules, sequesters lymphocytes in lymph nodes, preventing them from contributing to an autoimmune reaction.

CSIMs also target and inhibit or modulate proteins such as certain enzymes found in (or on) immune cells.

One representative target enzyme is a Janus kinase (JAK) enzyme. These enzymes are involved in the JAK-STAT signaling pathway that transmits extracellular information into the cell nucleus, thereby influencing DNA transcription).

Another target enzyme is calcineurin (a calcium-dependent serine/threonine protein phosphatase that activates nuclear factor of activated T cell cytoplasmic (NFATc), a transcription factor.

Yet another target enzyme is mammalian target of rapamycin (mTOR), a member of the phosphatidylinositol 3-kinase-related kinase family of protein kinases family that regulates a variety of cell physiology including cell growth, cell proliferation, cell motility, cell survival, protein synthesis, autophagy, and transcription.

Other enzymes that are targets of cytostatic agents include dihydrofolate reductase (DHFR), which is involved in the synthesis of tetrahydrofolate; enzymes involved in purine synthesis and metabolism including adenylosuccinate synthase, adenylosuccinate lyase, inosine-monophosphate dehydrogenase, GMP synthase, glutamine-phosphoribosyl pyrophosphate amidotransferase (GPAT), etc.); and dihydroorotate dehydrogenase (DHODH), a mitochondrial enzyme involved in the de novo synthesis of uridine monophosphate (UMP), which is required for the synthesis of DNA and RNA.

### CSIMS

The CSIMs of the present disclosure achieve their uniqueness from their structure. The CSIMs include two components: (1) a “bait” or selective moiety, which is a substrate that can be cleaved, e.g., by an enzyme that is highly expressed in the target cell type; and (2) a “payload” moiety, which is the active inhibitor or modulator of an enzyme or receptor found in immune cells and is an antiproliferative and immunomodulatory agent.

### Bait—Payload

The payload of the CSIMs comprise a fully active moiety which is a receptor inhibitor/modulator or enzyme inhibitor/modulator and which does not require a conversion step to become therapeutic. Thus, CSIMs comprise active payloads, including the active species of a normally inactive payload compound that naturally gets metabolically converted to the active species within the body. The CSIMs can also comprise a linker moiety which links the bait to the payload, and which, by itself, is not active and may be unstable.

The bait is a molecule comprising about 1-5 units which can be one or multiples of the same or a combination of different small organic molecules such a, but not limited to, amino acids, sugars, and/or lipids, including, but not limited to, those existing in nature. This component optimizes the CSIMs to achieve immune cell-selective accumulation by serving, for example, as a substrate for one or more enzymes present in the targeted cells or which binds to a receptor found on the targeted cells. It also serves to stabilize the CSIM compound, assisting in inhibiting the activity of the payload by restricting cell permeability or by electrostatic or steric interference with regions on the target, for example. Thus, the activity of the payload in the CSIM is inhibited or inactive until the bait component is released.

Cleavage of the bait or selectivity moiety and release of the payload occurs when disease factors are present, thus sparing unaffected cells. As used herein, the term “disease factors” encompasses compounds and molecules present in the environment of the cancer or affected cells and which are associated with the cancer or affected cells, and may be synthesized or elicited by the cancer or affected cells, or the presence thereof. Representative, nonlimiting disease factors include small molecules (chemical compounds) or macromolecules that circulate in plasma of a subject with active proliferative diseases, but not in healthy subjects or subjects that have their diseases in remission. The presence of a disease factor results in the production or activation of the enzyme or receptor that causes or results in the cleavage of the bait from the CSIM.

Representative baits are molecules or oligomers comprising about 1-5 units, which are, independently at each occurrence selected from the group consisting of

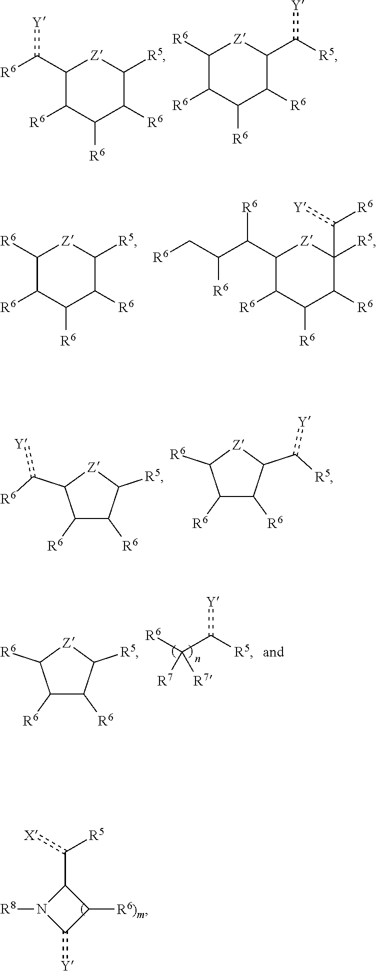

wherein:

Z′ is independently, at each occurrence, selected from the group consisting of CH₂, NH, O, and S;

X′ and Y′ are independently, at each occurrence, selected from the group consisting of O, NH, and S;

R⁵ is independently, at each occurrence, selected from the group consisting of a bond to any R⁶, a bond to any R⁸, and a bond to the payload;

R⁶ is independently, at each occurrence, selected from the group consisting of H, —R^{D}, —R^{A}—R⁵, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};

R^{A} is independently, at each occurrence, selected from the group consisting of CH₂, NH, O, and S;

R^{B} is independently, at each occurrence, selected from the group consisting of O, NH, and S;

R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;

R^{D} is independently, at each occurrence, selected from the group consisting of OH, SH, NH₂, N₃, and halo;

R⁷ and R^{7′} are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;

R⁸ is independently, at each occurrence, a bond to R⁵;

m is 0, 1, 2, 3, or 4; and

n is 0, 1, 2, or 3.

Useful payloads include, but are not limited to those payloads, or their metabolically active species, which modulate certain immune cell receptors such as, but not limited to, the glucocorticoid receptor and the sphingosine-1-phosphate receptor (S1P) receptor. Other useful payloads are those which modulate certain enzymes such as, but not limited to, a Janus kinase, calcineurin, mammalian target of rapamycin (mTOR), dihydrofolate reductase (DHFR), and dihydroorotate dehydrogenase (DHODH). Yet other exemplary enzymes that a payload modulates are those involved in purine synthesis and metabolism, such as, but not limited to, adenylosuccinate synthase, adenylosuccinate lyase, inosine-monophosphate dehydrogenase, GMP synthase, and glutamine-phosphoribosyl pyrophosphate amidotransferase (GPAT).).

Exemplary useful CSIMs comprising corticosteroids as payloads that have the structures of Formulae I-III below.

A CSIM having the structure of Formula I comprises:

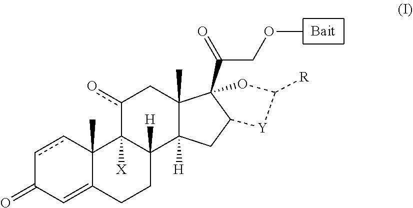

and pharmaceutically acceptable salts thereof, wherein:
[symbol] is an optional bond;
R is selected from the group consisting of H, C₁₋₄ alkyl, C₂₋₄ alkenyl, C₂₋₄ vinyl, and C₃₋₄ allyl;
X is selected from the group consisting of hallo, and H; and
Y is selected from the group consisting of CH₂ O, and NH.

Representative CSIMs having the structure of Formula I include:

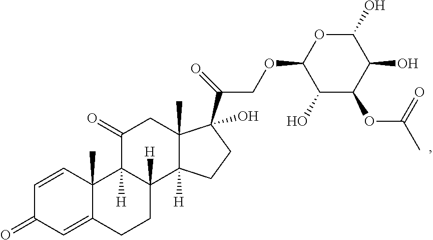

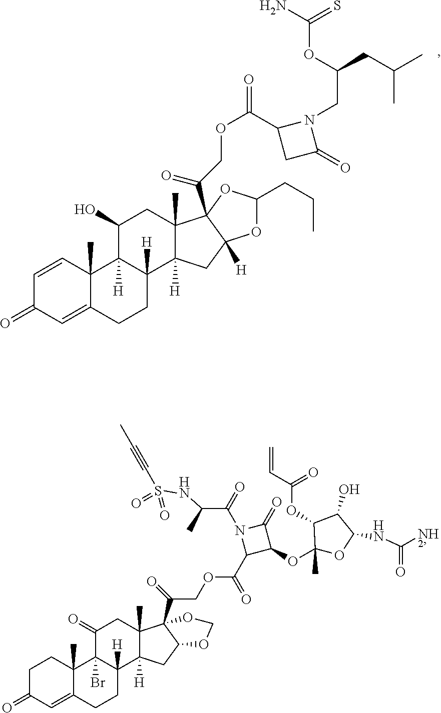

and pharmaceutically acceptable salts thereof.

A CSIM alternatively has the structure of Formula II:

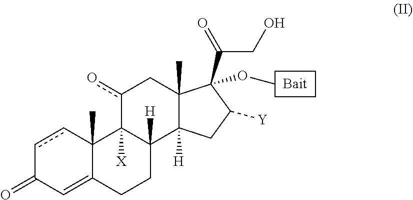

and pharmaceutically acceptable salts thereof, wherein:
[symbol] is an optional bond;
R is selected from the group consisting of H, C₁₋₄ alkyl, C₂₋₄ alkenyl, C₂₋₄ vinyl, and C₃₋₄ allyl;
X is selected from the group consisting of hallo, and H; and
Y is selected from the group consisting of CH₂ O, and NH.

Representative CSIMs having the structure of Formula II include:

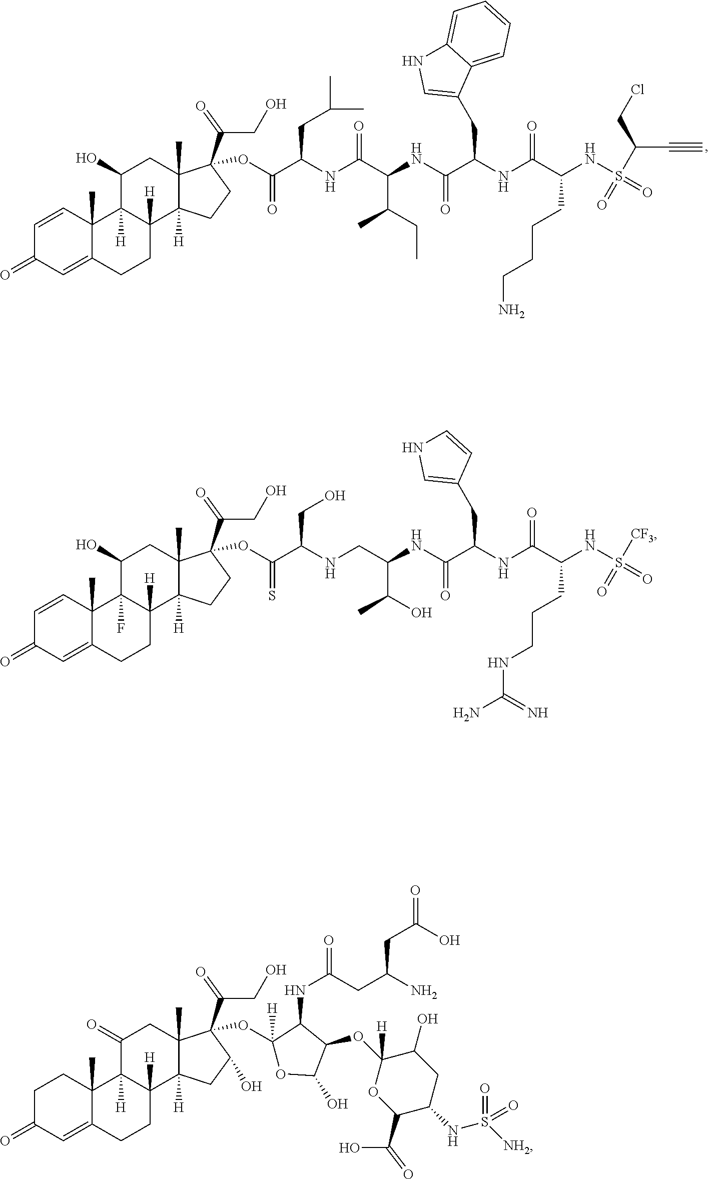

and pharmaceutically acceptable salts thereof.

Alternatively, a CSIM has the structure of Formula III:

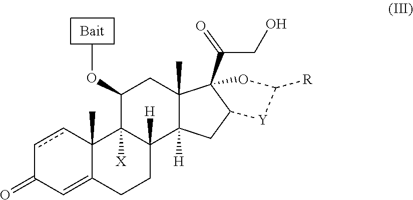

and pharmaceutically acceptable salt thereof, wherein:
[symbol] is an optional bond;
R is selected from the group consisting of H, C₁₋₄ alkyl, C₂₋₄ alkenyl, C₂₋₄ vinyl, and C₃₋₄ allyl;
X is selected from the group consisting of hallo, and H; and
Y is selected from the group consisting of CH₂ O, and NH.

Representative CSIMs having the structure of Formula III include:

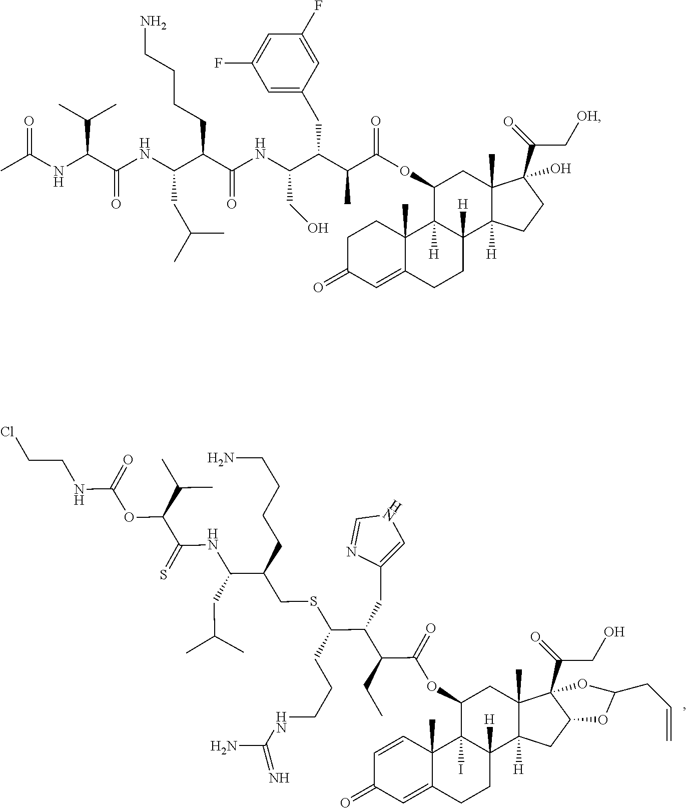

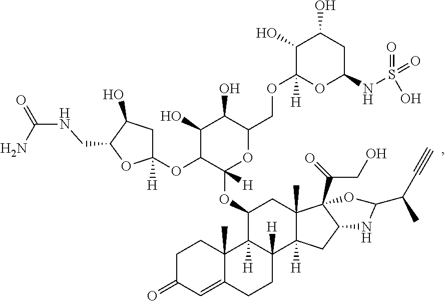

and pharmaceutically acceptable salts thereof.

Exemplary useful CSIMs comprising payloads including Janus kinase (JAK) inhibitors fall into the generic structures of Formulae IV-VIII below.

A CSIMs having Formula IV comprises:

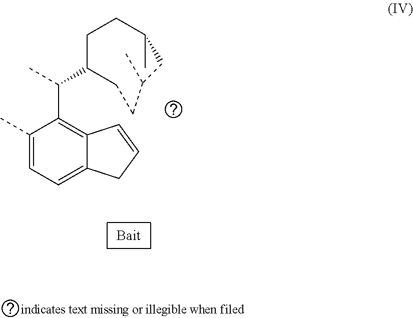

and pharmaceutically acceptable salts thereof, wherein:
[symbol] is an optional bond;
W at each instance is individually selected from the group consisting of Nitrile (CN). Sulfonamide (SO₂NHR for R═H, C₁₋₄ alkyl, C₂₋₄ alkenyl, C₂₋₄ vinyl, and C₃₋₄ allyl), or amide (CONR for SO₂NHR for R═H, C₁₋₄ alkyl, C₂₋₄ alkenyl, C₂₋₄ vinyl, and C₃₋₄ allyl) for each instance; and
Y is selected from the group consisting of C, and N.

Representative CSIMs with structures of Formula IV include:

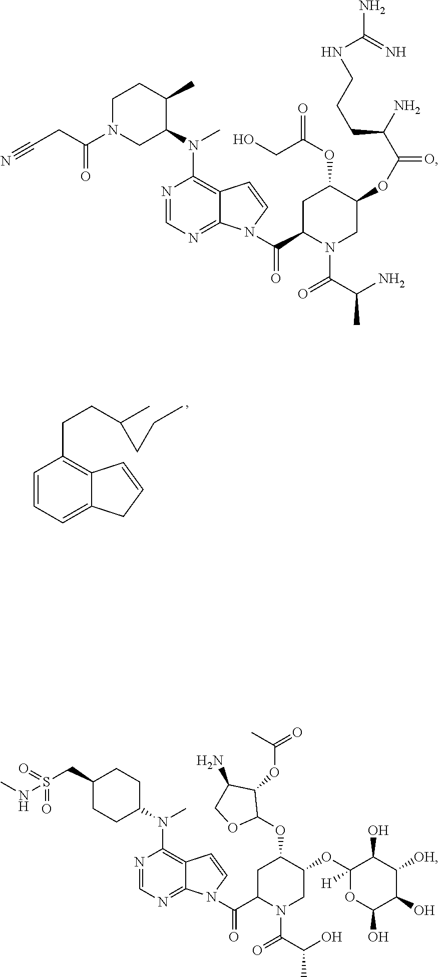

and pharmaceutically salt thereof.

A CSIMs having Formula V comprises:

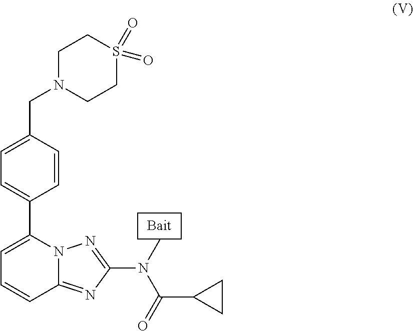 [SMILES: CN(C(=O)C1CC1)c1nc2cccc(-c3ccc(CN4CCS(=O)(=O)CC4)cc3)n2n1]

and pharmaceutically acceptable salts thereof.

Representative CSIMs having the structure of Formula V include:

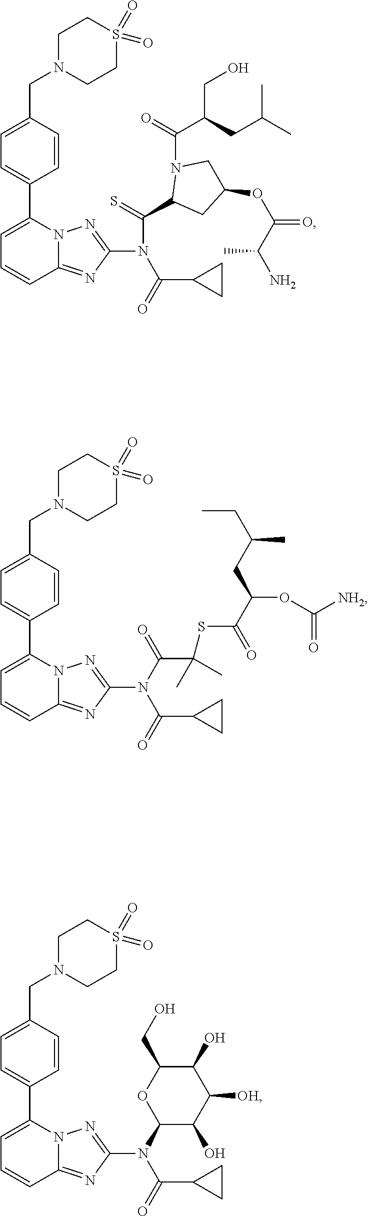

and pharmaceutically acceptable salts thereof.

A CSIMs having Formula VI comprises:

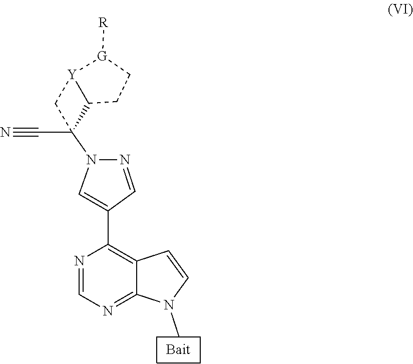

and pharmaceutically acceptable salts thereof, wherein:
[symbol] is an optional bond;
R is selected from the group consisting of H, C₁₋₄ alkyl, C₂₋₄ alkenyl, C₂₋₄ vinyl, and C₃₋₄ allyl;
G is selected from the group consisting of CH, —CO—, —SO—, and —SO₂—; and
Y is selected from the group consisting of CH, and N.

Representative CSIMs having the structure of Formula VI include:

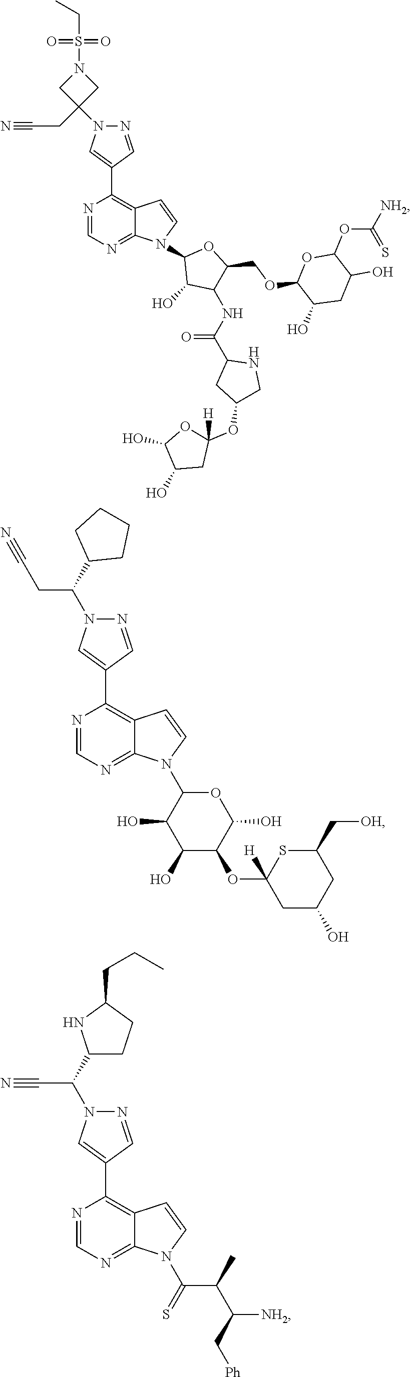

and pharmaceutically acceptable salts thereof.

A CSIM having the structure of Formula VII comprises:

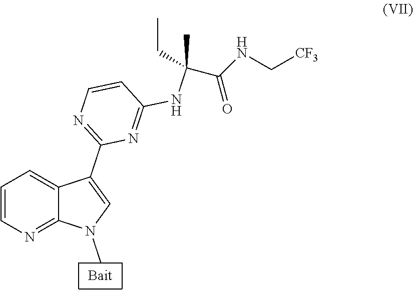 [SMILES: CC[C@@](C)(Nc1ccnc(-c2cn(C)c3ncccc23)n1)C(=O)NCC(F)(F)F]

and pharmaceutically acceptable salts thereof.

Representative CSIMs having the structure of Formula VII include:

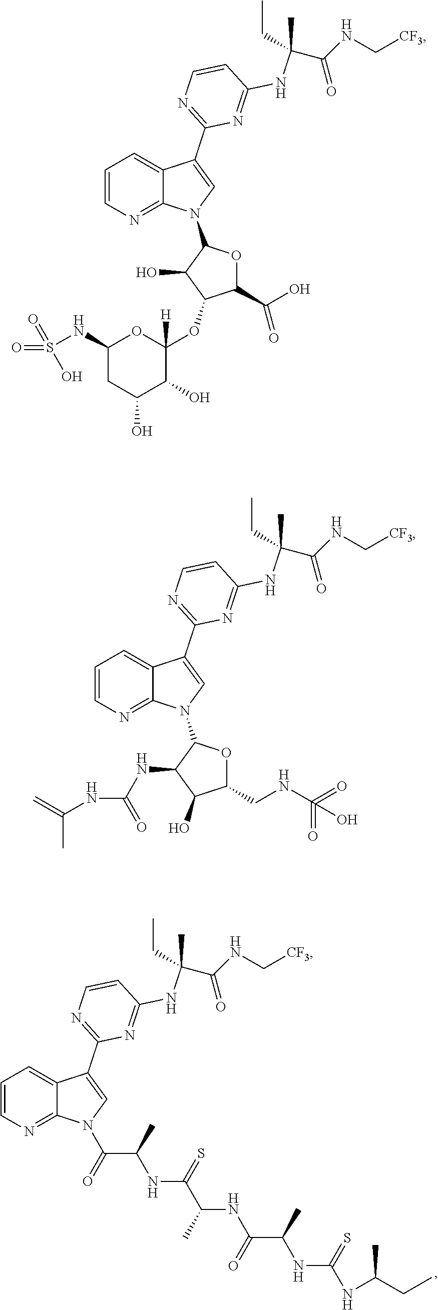

and pharmaceutically acceptable salts thereof.

A CSIM having the structure of Formula VIII comprises:

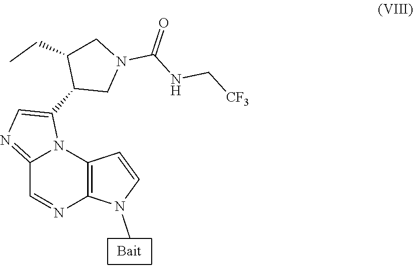 [SMILES: CC[C@@H]1CN(C(=O)NCC(F)(F)F)C[C@@H]1c1cnc2cnc3c(ccn3C)n12]

and pharmaceutically acceptable salts thereof.

Representative CSIMs having the structure of Formula VIII include:

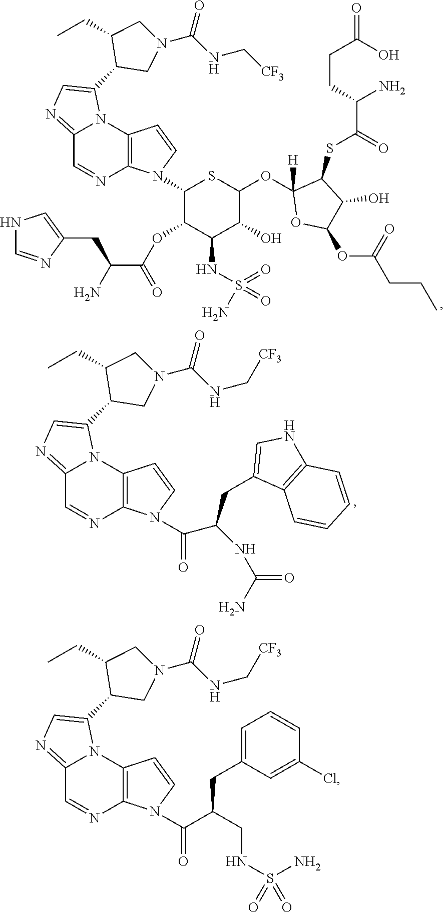

and pharmaceutically acceptable salt thereof.

Exemplary useful metabolically activated Calcineurin inhibitors fall into the generic structures of Formulae IX-XI below.

A CSIM of Formula IX comprises:

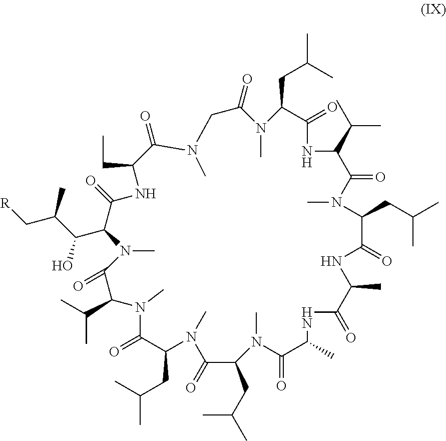

and pharmaceutically acceptable salts thereof, wherein R is selected from the group consisting of C₁₋₈ alkyl, C₂₋₈ alkenyl, C₂₋₈ vinyl, and C₃₋₈ allyl.

Representative CSIMs having the structure of Formula IX include:

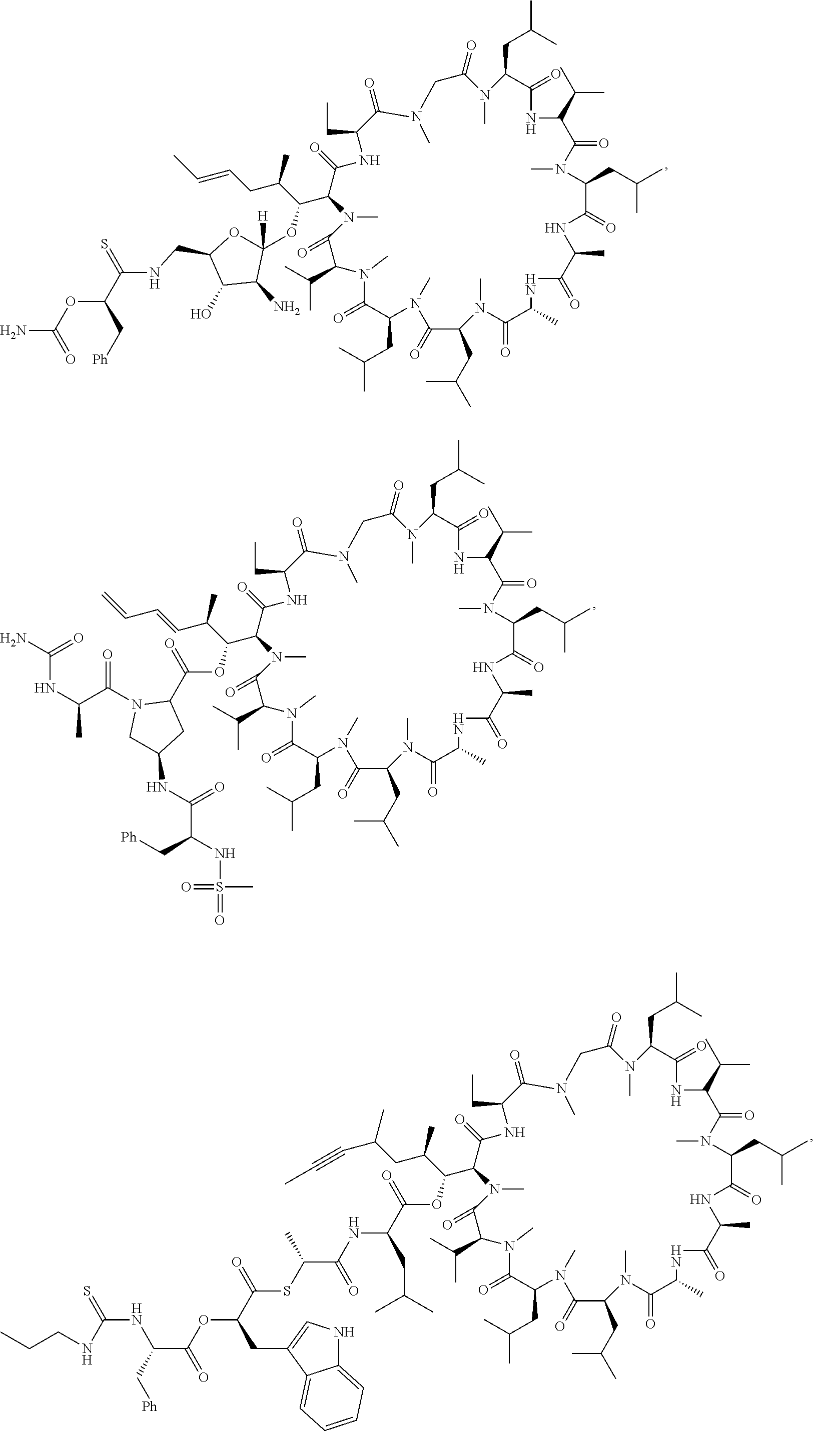

and pharmaceutically acceptable salts thereof.

A CSIM having the structure of Formula X comprises:

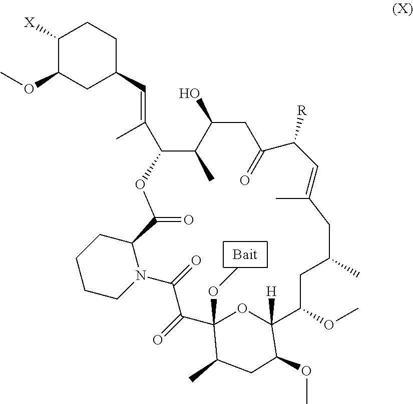

and pharmaceutically acceptable salts thereof, wherein:
R is selected from the group consisting of H, C₁₋₆ alkyl, C₂₋₆ alkenyl, C₂₋₆ vinyl, and C₃₋₆ allyl; and
X is selected from the group consisting of hallo, and OH.

Representative CSIMs having the structure of Formula X include:

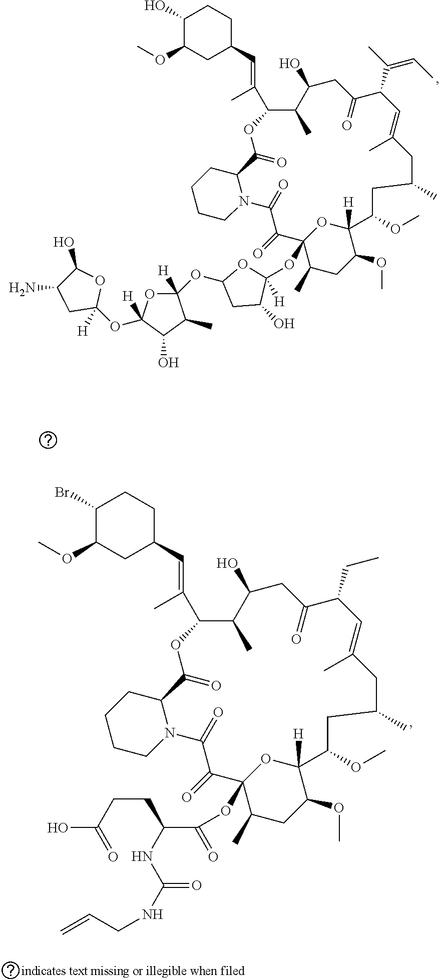

and pharmaceutically acceptable salts thereof.

A CSIM having the structure of Formula XI comprises:

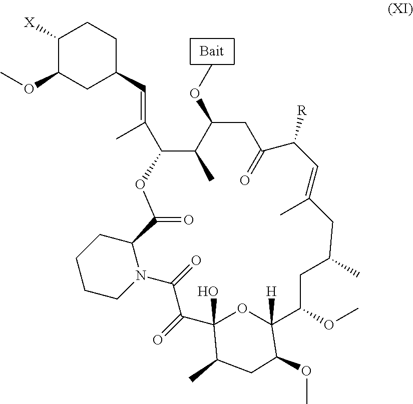

and pharmaceutically acceptable salts thereof, wherein:
R is selected from the group consisting of H, C₁₋₆ alkyl, C₂₋₆ alkenyl, C₂₋₆ vinyl, and C₃₋₆ allyl; and
X is selected from the group consisting of hallo, and OH;

Representative CSIMs having the structure of Formula XI include:

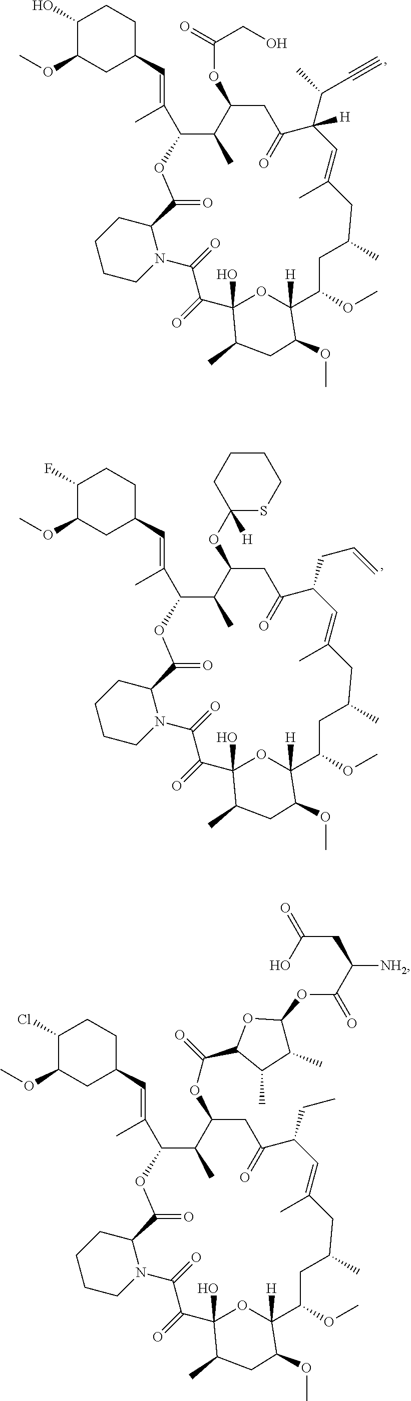

and pharmaceutically acceptable salts thereof.

Exemplary useful metabolically activated mTOR inhibitors fall into the generic structures of Formulae XII and XIII below.

A CSIM having the structure of Formula XII comprises:

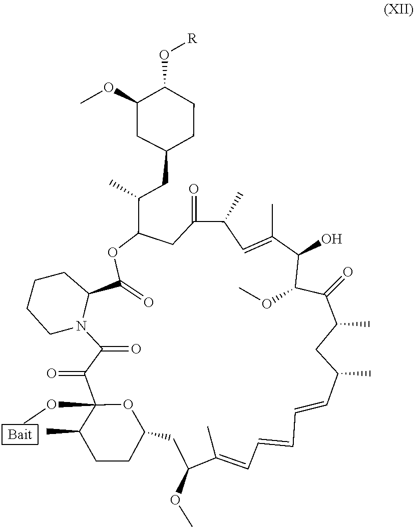

and pharmaceutically acceptable salts thereof, wherein R is selected from the group consisting of either H, or one of the following groups with an OH substituent; C₁₋₈ alkyl, C₂₋₈ alkenyl, C₂₋₈ vinyl, and C₃₋₈ allyl.

Representative CSIMs having the structure of Formula XII include:

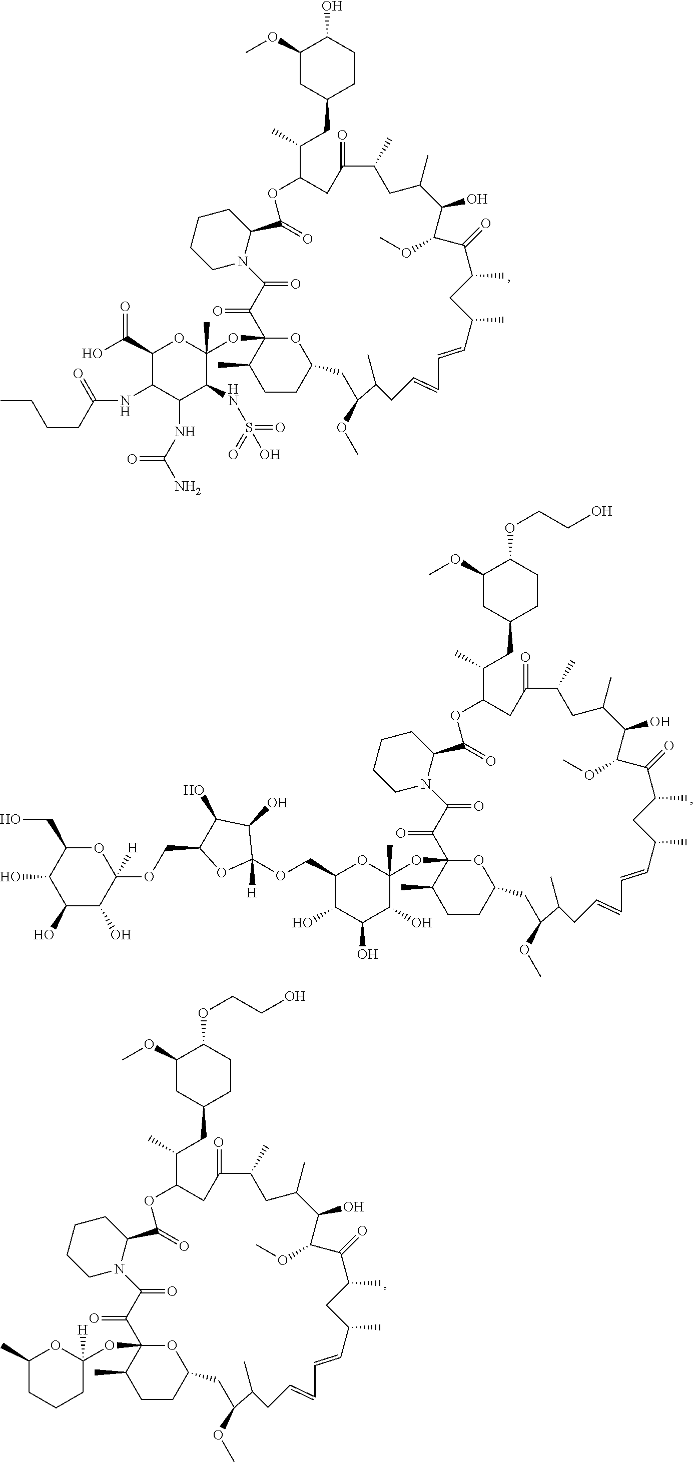

and pharmaceutically acceptable salts thereof.

A CSIM [having the structure of Formula XIII comprises:

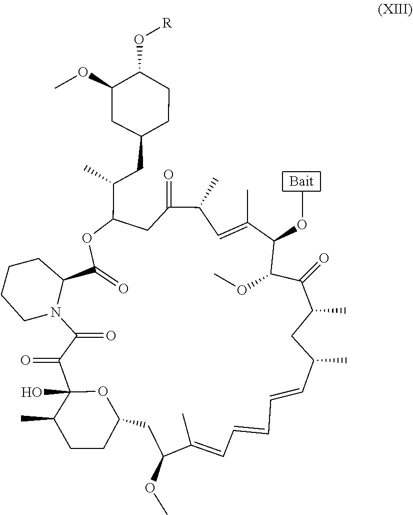

and pharmaceutically acceptable salts thereof, wherein R is selected from the group consisting of either H, or one of the following groups with an OH substituent; C₁₋₈ alkyl, C₂₋₈ alkenyl, C₂₋₈ vinyl, and C₃₋₈ allyl.

Representative CSIMs having the structure of Formulas XIII include:

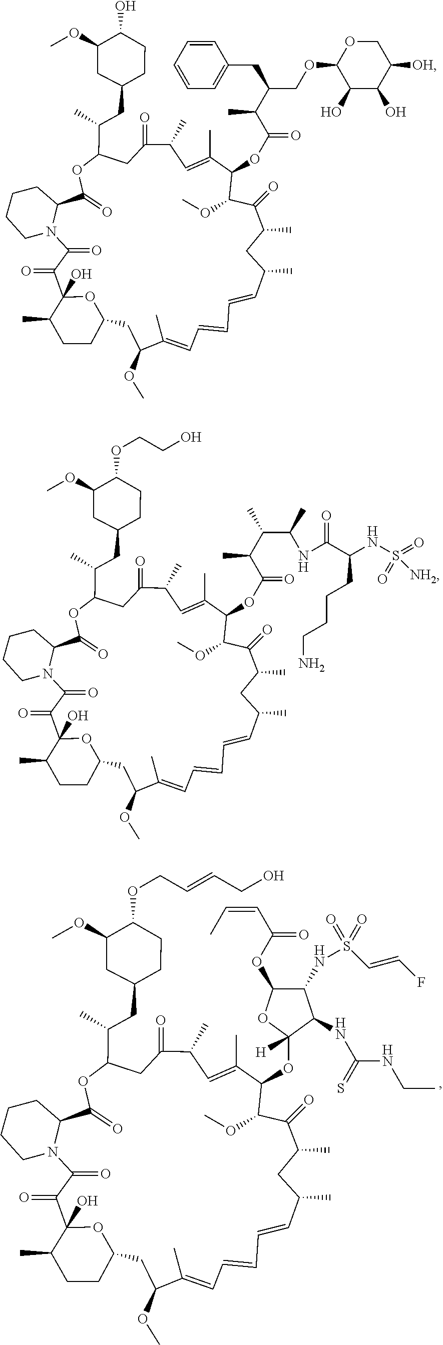

and pharmaceutically acceptable salts thereof.

A CSIM that modulate the sphingosine-1-phosphate receptor having the structure of Formula XIV comprises:

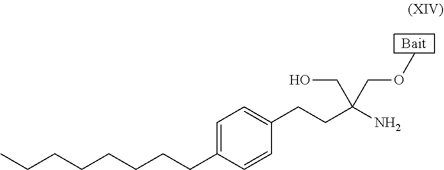 [SMILES: CCCCCCCCc1ccc(CCC(N)(CO)COC)cc1]

and pharmaceutically acceptable salts thereof.

Representative CSIMs having the structure of Formula XIV include:

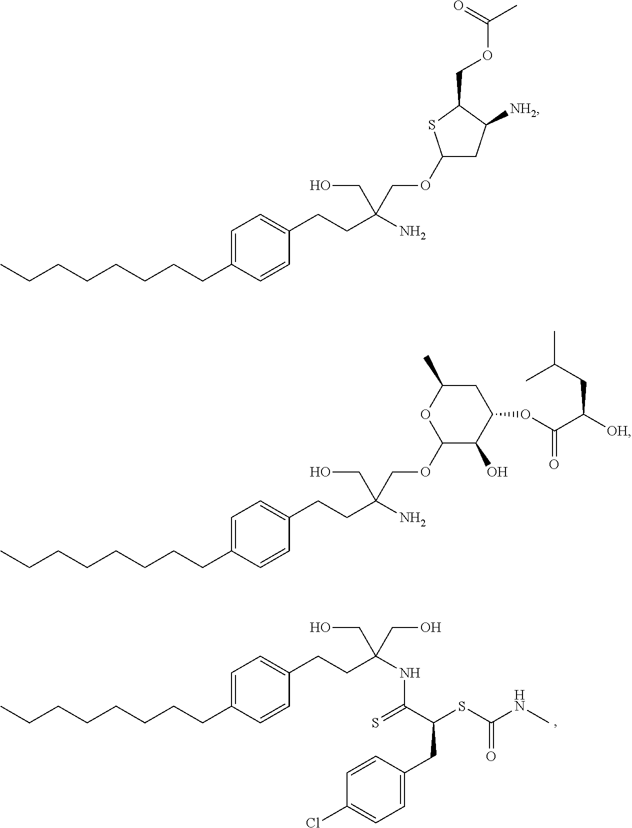

and pharmaceutically acceptable salts thereof.

A CSIM that modulate the sphingosine-1-phosphate receptor having the structure of Formula XV comprises:

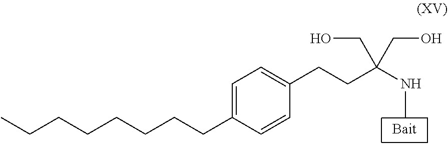 [SMILES: CCCCCCCCc1ccc(CCC(CO)(CO)NC)cc1]

and pharmaceutically acceptable salts thereof.

Representative CSIMs having the structure of Formula XV include:

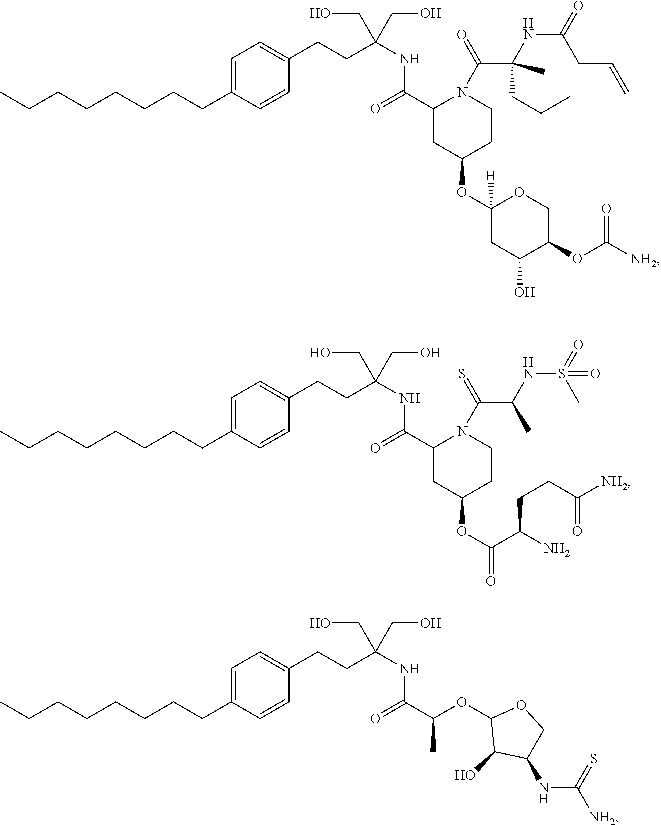 [SMILES: C=CCC(=O)N[C@](C)(CCC)C(=O)N1CC[C@@H](O[C@@H]2C[C@@H](O)[C@H](OC(N)=O)CO2)CC1C(=O)NC(CO)(CO)CCc1ccc(CCCCCCCC)cc1 ; CCCCCCCCc1ccc(CCC(CO)(CO)NC(=O)C2C[C@H](OC(=O)[C@H](N)CCC(N)=O)CCN2C(=S)[C@H](C)NS(C)(=O)=O)cc1 ; CCCCCCCCc1ccc(CCC(CO)(CO)NC(=O)[C@H](C)OC2OC[C@@H](NC(N)=S)[C@H]2O)cc1]

and pharmaceutically acceptable salts thereof.

A CSIM that modulate the sphingosine-1-phosphate receptor having the structure of Formula XVI comprises:

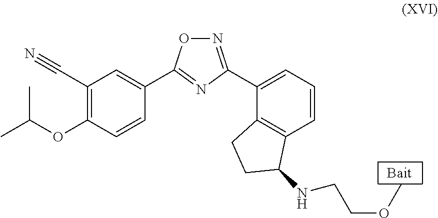 [SMILES: COCCN[C@H]1CCc2c(-c3noc(-c4ccc(OC(C)C)c(C#N)c4)n3)cccc21]

and pharmaceutically acceptable salts thereof.

Representative CSIMs have the structure of Formula XVI include:

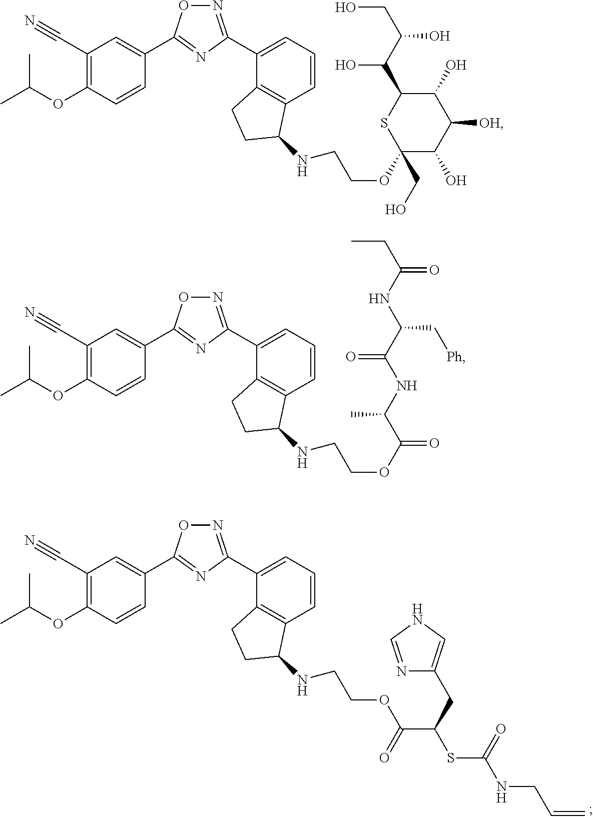

and pharmaceutically acceptable salts thereof.

A CSIM that modulate the sphingosine-1-phosphate receptor having the structure of Formula XVII comprises:

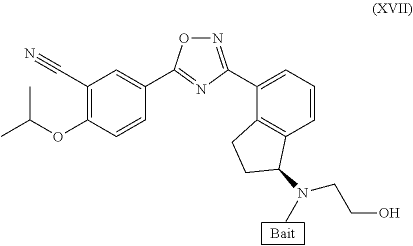 [SMILES: CC(C)Oc1ccc(-c2nc(-c3cccc4c3CC[C@@H]4N(C)CCO)no2)cc1C#N]

and pharmaceutically acceptable salts thereof.

Representative CSIMs having the structure of Formula XVII include:

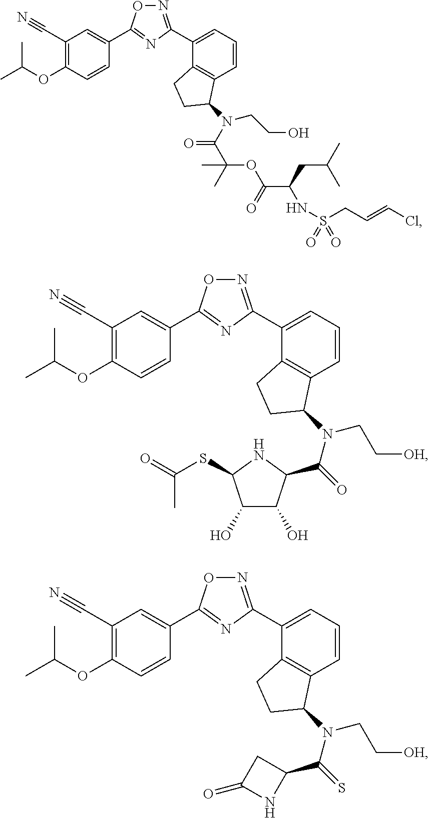

and pharmaceutically acceptable salts thereof.

A CSIM that modulate the sphingosine-1-phosphate receptor having the structure of Formula XVIII comprises:

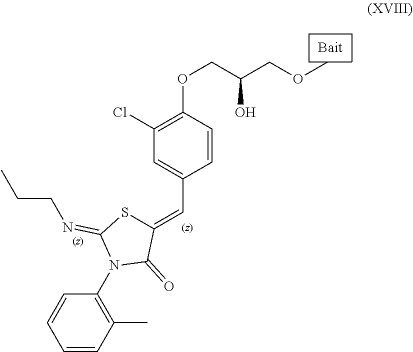 [SMILES: CCC/N=C1\S/C(=C\c2ccc(OC[C@H](O)COC)c(Cl)c2)C(=O)N1c1ccccc1C]

and pharmaceutically acceptable salts thereof.

Representative CSIMs having the structure of Formula XVIII include:

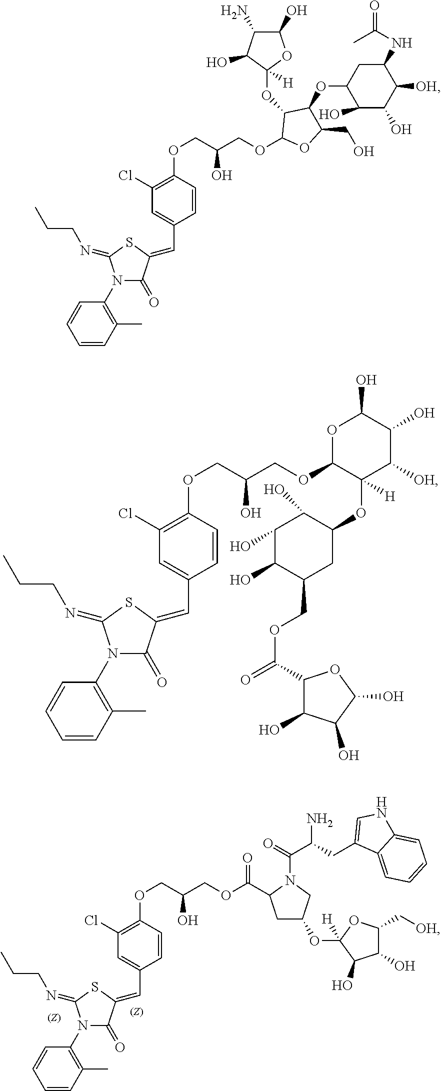

and pharmaceutically acceptable salts thereof.

A CSIM that modulate the sphingosine-1-phosphate receptor having the structure of Formula XIX comprises:

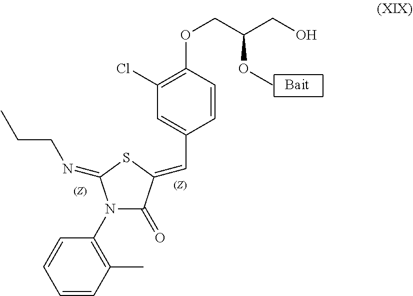 [SMILES: CCC/N=C1\S/C(=C\c2ccc(OC[C@@H](CO)OC)c(Cl)c2)C(=O)N1c1ccccc1C]

and pharmaceutically acceptable salts thereof.

Representative CSIMs having the structure of Formula XIX include:

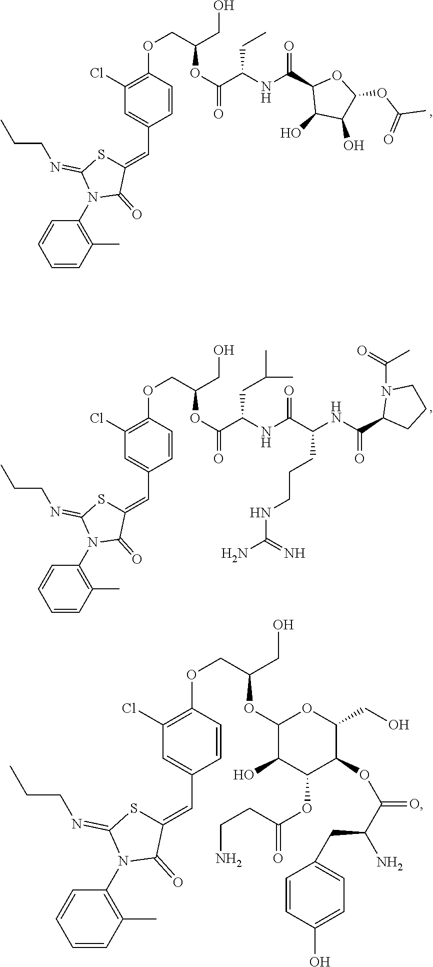

and pharmaceutically acceptable salts thereof.

The CSIM compounds described herein can be asymmetric (e.g., having one or more stereocenters). All stereoisomers of such compounds, such as enantiomers and diasteromers, are intended unless otherwise indicated.

CSIM compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis.

Many geometric isomers of olefins, C═N double bonds and the like can also be present in the CSIM compounds described herein, and all such stable isomers are contemplated in the present disclosure. Cis and trans geometric isomers of the CSIM compounds of the present disclosure are described and may be isolated as a mixture of isomers or as separated isomeric forms.

Resolution of racemic mixtures of CSIM compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as +/−camphorsulfonic acid. Other resolving agents suitable for fractional crystallization methods include stereoisomerically pure forms of +/−methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

The CSIM compounds of the present disclosure can have the (R)-configuration or the (S)-configuration. In compounds with more than one chiral center, each of the chiral centers in the compound may be independently (R) or (S), unless otherwise indicated.

CSIM compounds according to the present disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

### Syntheses

Synthesis of a CSIM compound according to the disclosure includes any method known to one with skill in the art. One representative method is as follows.

Payloads as described herein can be commercially obtained or synthesized by alterations of methods known to one with skill in the art, for example, from their commercially available components via existing reported methods.

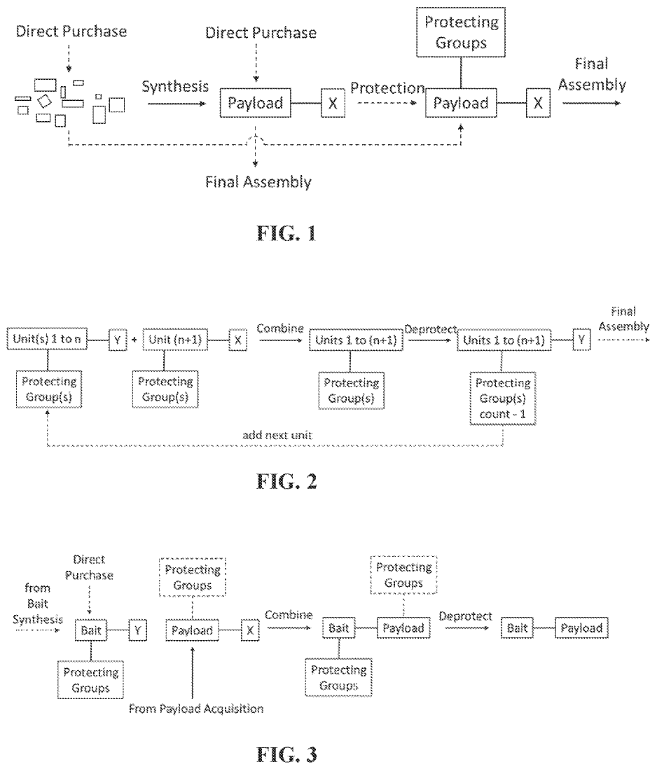

As shown in FIG. 1, payloads present a functional group (labeled “X”), and specified by the heteroatom used to attach to “Bait” in the generic structures of the CSIMs described herein. Payloads that present only one exposed functional position that is potentially active for the coupling reaction necessary to attach the Bait can be brought directly onto the Final Assembly process (FIG. 3). Payloads with multiple functional groups that are potentially reactive or chemically sensitive to the Bait coupling chemistry in the Final Assembly process (FIG. 3) may include other potentially active or reactive substrates that need to be protected first. This may be done directly with the completed Payload via standard means (e.g., Wuts and Green; Greene's Protective Groups in Organic Synthesis, Fourth Edition; John Wiley & Sons, Inc.: 2007), or can be done directly from altering the synthesis of the payload, e.g., by omitting steps that remove protecting groups in the synthesis itself as well as adding, or by changing the protecting groups specified in the existing synthesis as necessary.

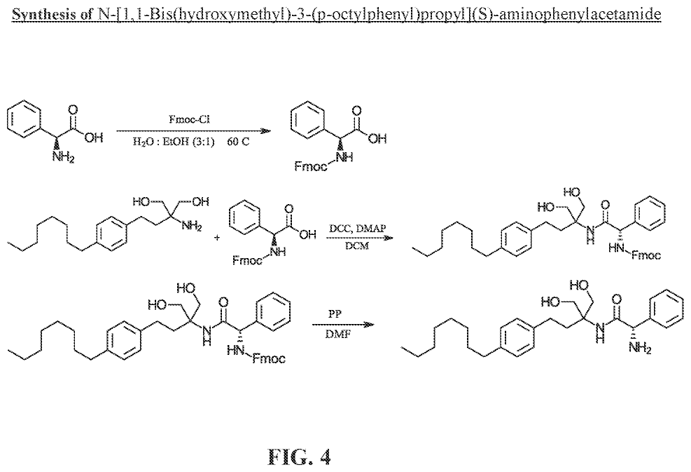

Bait components may be obtained commercially or synthesized by methods known by those with skill in the art; for Bait components that are not commercially available as ready units for use in the final assembly (e.g. Fmoc-Gly-Tyr(tBu)-OH a.k.a. Pubchem ID=329767849), Baits are individually synthesized from amino acid and/or sugar components via standard methods (e.g., Jaradat (2017) Amino Acids. 50 (1); 39-68) and/or oligosaccharide synthesis (e.g., Levy et al. The Organic Chemistry of Sugar; Taylor & Francis: 2006) depending on the composition of a given bait. When Baits are built up from commercially available units, this is done in a sequential manner of coupling and deprotecting as generically outlined in FIG. 2. For a bait to be ready to be attached to either a Linker or a Payload directly, a single R5 position that is the point of attachment to the linker or payload is left un-protected (labeled as “Y” in the figure below,) while all other aliphatic amines and protic functionality with a pKa <20 in DMSO are protected via standard means (see, e.g., Wuts and Green (2007) supra).

The final assembly (FIG. 3) is carried out by coupling the appropriately protected Bait, either from the Bait synthesis as described supra, or commercially obtained, and the Payload with or without protecting groups as necessary described supra. This is done with an appropriate coupling reaction used for the exposed functionality on both the Payload and Bait (e.g., a “Bait” presenting a “Y”, a.k.a. R⁵, with a carboxyl group may be coupled to a “Payload” presenting an “X” that was an aliphatic amine with N,N′-dicyclohexylcarbodiimide (DCC)). All residual protecting groups are then removed from the resulting assembled compound to yield the active final “Bait”-“Payload” or “Bait”-“Linker”-“Payload” assembly.

### Disorders to be Treated

The CSIMs according to the disclosure are useful for treating proliferative disorders impacting immune cells.

For example, CSIMS with glucocorticoid receptor inhibitor payloads are used to treat autoimmune diseases and conditions such as allergy and asthma, or in transplant medicine to prevent acute transplant rejection or graft-versus-host disease.

CSIMS with JAK inhibitor payloads are used to treat rheumatoid arthritis, psoriatic arthritis, ankylosing spondyliti, ulcerative colitis, psoriasis, alopecia areata, vitiligo and atopic dermatitis.

CSIMS with calcineurin inhibitor payloads are used to treat rheumatoid arthritis, psoriatic arthritis, psoriasis, acute ocular Behçet's disease, juvenile idiopathic arthritis, adult and juvenile polymyositis and dermatomyositis, adult and juvenile systemic lupus erythematosus, adult lupus membranous nephritis, systemic sclerosis, aplastic anemia, steroid-resistant nephrotic syndrome, atopic dermatitis, severe corticosteroid-dependent asthma, severe ulcerative colitis, pemphigus vulgaris, myasthenia gravis, and dry eye disease, with or without Sjögren's syndrome (as ophthalmic emulsion).

CSIMS with mTOR inhibitor payloads are used to prevent kidney or liver transplant rejection and to treat lymphangioleiomyomatosis.

CSIMS with folic acid inhibitor payloads such as MTX are used to treat rheumatoid arthritis, juvenile dermatomyositis, psoriasis, psoriatic arthritis, lupus, multiple sclerosis, sarcoidosis, Crohn's disease, ulcerative colitis, eczema and many forms of vasculitis. It is also used to treat a range of cancers including Leukemia and Lymphoma.

CSIMS with purine analog payloads such as AZA and mercaptopurine are used to treat rheumatoid arthritis, multiple sclerosis, pemphigus, Behçet's disease, vasculitis, autoimmune hepatitis, atopic dermatitis, myasthenia gravis, neuromyelitis optica (Devic's disease), restrictive lung disease, idiopathic pulmonary fibrosis, granulomatosis with polyangiitis, Crohn's disease, ulcerative colitis, systemic lupus erythematosus, and in prevention of kidney transplants rejection. They can also be used to treat acute lymphocytic leukemia (ALL) and chronic myeloid leukemia (CML).

CSIMS with DHODH inhibitor payloads such as leflunomide (Arava), brequinar are used to treat rheumatoid arthritis and psoriatic arthritis, and potentially Crohn's disease, sarcoidosis, uveitis, Still's disease, pemphigoid, Kimura's disease, systemic lupus erythematosus, Felty's syndrome, Takayasu arteritis, granulomatosis with polyangiitis, ankylosing spondylitis and prevention of organ transplant rejections.

CSIMS with S1P receptor modulator payloads are used to treat multiple sclerosis and potentially psoriasis.

Other proliferative disorders that can be treated are inflammatory diseases such as atherosclerosis (AS), where macrophages play a central role. Asthma (involving T cells, macrophages, neutrophils, cosinophils) can also be treated.

Particular autoimmune diseases are also treatable with these inhibitors. For example, rheumatoid arthritis (RA), inflammatory bowel diseases (IBD), celiac disease, diabetes mellitus type 1, Graves' disease, multiple sclerosis (MS), systemic lupus erythematosus (SLE). Sarcoidosis, pemphigus vulgaris (mostly B cell driven), myasthenia gravis (mostly B cell driven), and psoriasis can be treated.

### Pharmaceutical Formulations and Treatment

The CSIMs according to the disclosure are useful in in pharmaceutical formulations that treat proliferative disorders. These pharmaceutical formulations include a therapeutically effective amount of a CSIM which has anti-proliferative and immunomodulatory properties a pharmaceutically acceptable carrier. The formulations may also comprise two or more different CSIM compounds. For example, the formulation can comprise a CSIM compound with one payload and a different CSIM with a different payload or different payload and different bait. The formulations according to the disclosure may also comprise another cancer therapeutic different that the CSIMs of the present disclosure. Nonlimiting, exemplary therapeutic agents include TNF inhibitors, IL-6 inhibitor, IL-12 inhibitor, BLyS inhibitor CTLA4-IgGs and anti-CD20 antibodies

The term “pharmaceutically acceptable carrier” is to be understood herein as referring to any substance that may, medically, be acceptably administered to a patient, together with ta derivative according to the disclosure, and which does not undesirably affect the pharmacological and synergistic activity of the inhibitor. A “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington, The Science and Practice of Pharmacy (9th Ed. 1995).

The CSIMs of the present disclosure can also be in the form of conventional, non-toxic salts which can be prepared, for example, from non-toxic inorganic or organic acids. The pharmaceutically acceptable CSII salts of the present disclosure can be synthesized from the parent CSII which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as, but no limited to, ether, ethyl acetate, ethanol, isopropanol, or acetonitrile are useful. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences (7th ed., Mack Publishing Company, Easton. Pa., 1985, p. 1418; and J. Pharm. Sci. (1977) 66: 2).

For use in medicine, the salts of the anti-proliferative CSIMs are pharmaceutically acceptable salts. Other salts may, however, be useful in the preparation of the CSIIs or of their pharmaceutically acceptable salts according to the disclosure. Suitable pharmaceutical-salts of the CSIMs according to the present disclosure include acid addition salts which may, for example, be formed by mixing a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Additionally, where the anti-infective CSIMs in the formulation carries an acidic moiety, suitable pharmaceutically acceptable salts thereof may include alkali metal salts, e.g., sodium or potassium salts; alkaline earth metal salts, e.g. calcium or magnesium salts; and salts formed with suitable organic ligands, e.g., quaternary ammonium salts.

The pharmaceutical formulations may be prepared for injectable use, topical use, oral use, intramuscular or intravenous injection, inhalation use, transdermal use, intradermal, transmembrane use, and the like. These formulations are in unit dosage forms such as tablets, pills, capsules, powders, granules, sterile parenteral solutions or suspensions, metered aerosol or liquid (nebulized) sprays, drops, ampoules, auto-injector devices or suppositories; for oral parenteral, intranasal, sublingual topical or rectal administration, or for administration by inhalation or insufflation. Alternatively, the formulations may be presented in a form suitable for one-weekly or once-monthly administration; for example, an insoluble salt of the derivative, such as decanoate salt, may be adapted to provide a depot preparation for intramuscular injection. An erodible polymer containing the inhibitor may also be envisaged.

For preparing solid compositions such as tablets, the CSIM is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a CSII of the present disclosure, or a pharmaceutically acceptable salt thereof.

These formulations may be homogeneous, i.e., the derivative is dispersed evenly throughout the composition so that the composition may be readily subdivided into equally effective unit dosage forms such as tablets, pills and capsules. This solid formulation composition is then subdivided into unit dosage forms of the type described above containing from about 0.1 mg to about 500 mg of the CSIM. Some useful unit dosage forms contain from 1 mg to 100 mg, for example, about 1 mg, about 2 mg, about 5 mg, about 10 mg, about 25 mg, about 50 mg, or about 100 mg, of the CSIM. The tablets or pills comprising the CSIMs can be coated or otherwise compounded to provide a dosage form affording the advantage of prolonged action. The liquid forms in which the CSIMs of the present disclosure may be incorporated for administration orally or by injection include aqueous solutions, suitably flavored syrups, aqueous or oil suspensions, and flavored emulsions with edible oils as well as elixirs and similar pharmaceutical vehicles.

Injectable dosage forms may be sterilized in a pharmaceutically acceptable fashion, for example by steam sterilization of an aqueous solution sealed in a vial under an inert gas atmosphere at 120° C. for about 15 minutes to 20 minutes, or by sterile filtration of a solution through a 0.2 μM or smaller pore-size filter, optionally followed by a lyophilization step, or by irradiation of a composition containing an inhibitor of the present disclosure by means of emissions from a radionuclide source.

A therapeutically effective dosage of the formulation according to the disclosure depends on the disorder treated and may vary from patient to patient, and factors such as the age and physical size of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 0.1 mg/kg to about 1000 mg/kg, or from about 1 mg/kg to about 100 mg/kg inhibitor may be suitable. For example, for the treatment of proliferation disorders, a suitable dosage level is about 0.001 mg/kg to about 250 mg/kg per day. The formulation maybe administered on bolus and or a regimen of about 1 to 4 times per day.

The CSIM formulations may additional comprise another anti-proliferative agent that is different than the CSIM compound. Nonlimiting, exemplary therapeutic agents include TNF inhibitors, IL-6 inhibitor, IL-12 inhibitor, BLyS inhibitor CTLA4-IgGs and anti-CD20 antibodies. The formulations may also comprise two or more different CSIM compounds, e.g., one with a methotrexate payload and one with a JAK inhibitor payloads.

Alternatively, when treating a subject with a CSIM formulation, treatment may also include administering another formulation comprising an anti-proliferative compound that is different than the CSIM formulation as set forth above.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Synthesis of N-[1,1-Bis(hydroxymethyl)-3-(p-octylphenyl)propyl](S)-aminophenylacetamide

The CSIM, N-[1,1-Bis(hydroxymethyl)-3-(p-octylphenyl)propyl](S)-aminophenyl-acetamide was prepared as shown in FIG. 5.

An unprotected bait, (S)-Aminophenylacetic acid, was protected with (9H-Fluoren-9-yl)methyl chloroformate (Fmoc chloride). To the mixture of the two solids, (S)-Aminophenylacetic acid (1 mmol) and Fmoc chloride (1.2 mmol), 1.5 mL of a 3:1 mixture of water to ethanol was added. The resulting solution was stirred at 60 C for 2 hours. The solution was acidified with HCl (1M) until pH 4-5 at 0 C and extracted with EtOAc (3×10 mL). The combined organic layers were merged dried over sodium sulfate and concentrated in vacuo to afford the protected bait, (S)-[(9H-Fluoren-9-yl)methoxycarbonyl-amino]phenylacetic acid. In this example the protected bait can also be purchased commercially from a number of vendors.

Thee protected Bait is coupled to the Payload, Fingolimod a.k.a. 2-Amino-2-[2-(p-octylphenyl)ethyl]-1,3-propanediol as follows. To a suspension of Fingolimod (1.13 mmol), 4-Dimethylaminopyridine (DMAP) (0.15 mmol) and the protected bait (1.47 mmol) in Dichloromethane (1.5 mL) at 0° C., was added N,N′-dicyclohexylcarbodiimide (DCC) in one portion (1.47 mmol). After 10 min the temperature was raised to 24 C and stirring was continued 2 hours. The reaction mixture was filtered and the precipitate washed with Dichloromethane (10 mL). The filtrate was washed with sat. NaHCO₃ (10 mL), dried with sodium sulfate and concentrated in vacuo to furnish the crude product, {(S)-[N-1,1-Bis(hydroxymethyl)-3-(p-octylphenyl)propylcarbamoyl]phenylmethyl}amino-(9H-fluoren-9-yl)methylformylate, which was purified by silica gel chromatography (30% EtOAc in hexanes).

The residual protecting groups are removed as follows. To a solution of {(S)-[N-1,1-Bis(hydroxymethyl)-3-(p-octylphenyl)propylcarbamoyl-]phenylmethyl}amino-(9H-fluoren-9-yl)methylformylate (1 mmol) in DMF (5 mL) was added piperidine (0.2 mmol). The solution was stirred at 24 C for 2 hours. The reaction was then extracted with ethyl acetate (2×10 mL), dried over sodium sulfate, and concentrated in vacuo, to yield the crude N-[1,1-Bis(hydroxymethyl)-3-(p-octylphenyl)propyl](S)-aminophenylacetamide. The final product was then purified by silica gel chromatography (1% methanol in dichloromethane).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. An immune cell-selective inhibitor or modulator (“CSIM”) compound having immunomodulatory or anti-proliferative properties, the CSIM comprising:
a bait comprising a molecule which target the CSIM to an immune cell, and
a payload comprising a small molecule modulator of an enzyme in, or a receptor on, the immune cell.

2. The compound of claim 1, comprising a bait comprising about 1 to about 5 units, which are, independently at each occurrence selected from the group consisting of 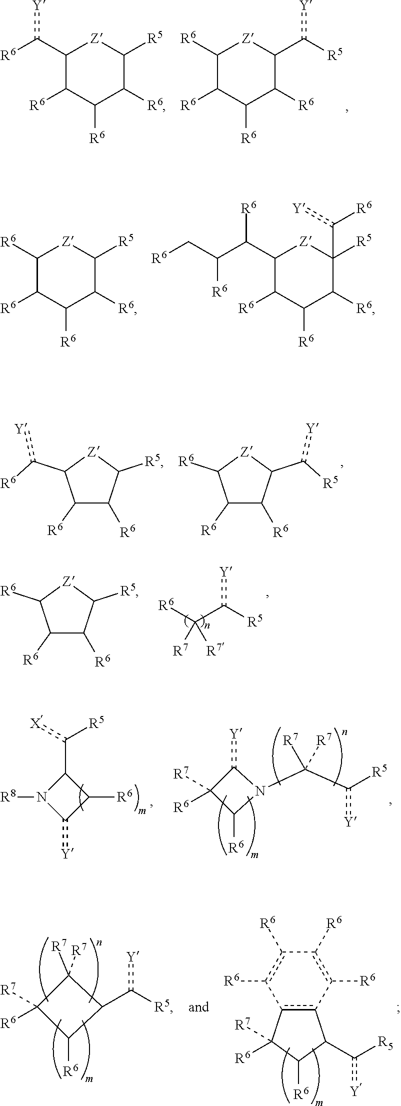
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and a bond to the payload;
R⁶ is independently, at each occurrence, selected from the group consisting of H, —R^{D}, —R^{A}—R⁵, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1, 2, or 3.

3. The compound of claim 2, wherein the payload targets the glucocorticoid receptor, the sphingosine-1-phosphate receptor (S1P) receptor, a Janus kinase, calcineurin, rapamycin (mTOR), dihydrofolate reductase (DHFR), dihydroorotate dehydrogenase (DHODH), purine synthesis and metabolism enzymes, adenylosuccinate synthase, adenylosuccinate lyase, inosine-monophosphate dehydrogenase, GMP synthase, or glutamine-phosphoribosyl pyrophosphate amidotransferase (GPAT).).

4. The compound of claim 2, comprising a payload comprising a corticosteroid.

5. The compound of claim 4, comprising a structure of Formula I: 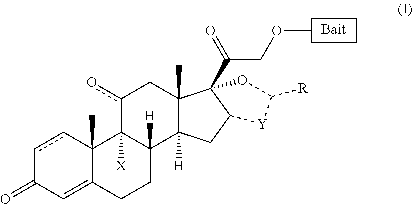
and pharmaceutically acceptable salts thereof, wherein: [symbol]
is an optional bond;
R is selected from the group consisting of H, C₁₋₄ alkyl, C₂₋₄ alkenyl, C₂₋₄ vinyl, and C₃₋₄ allyl;
X is selected from the group consisting of hallo, and H; and
Y is selected from the group consisting of CH₂ O, and NH.

6. The compound of claim 5, comprising the structure of Formula I, selected from the group consisting of 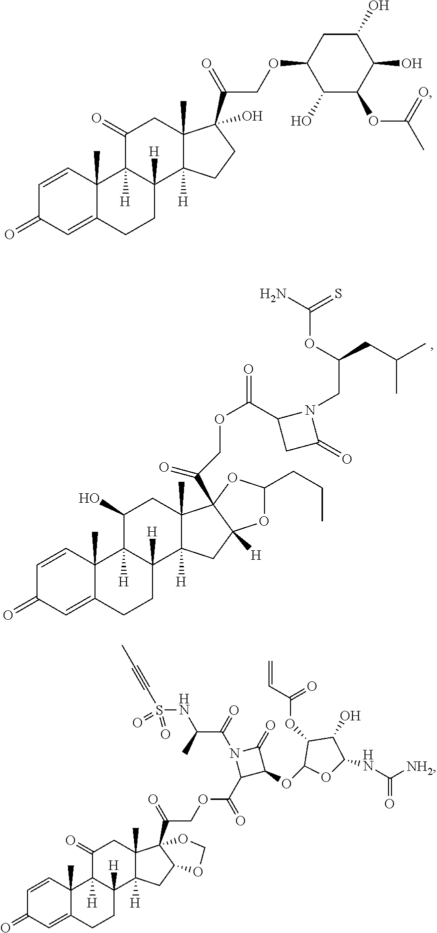
and pharmaceutically acceptable salts thereof.

7. The compound of claim 2, comprising the structure of Formula II: 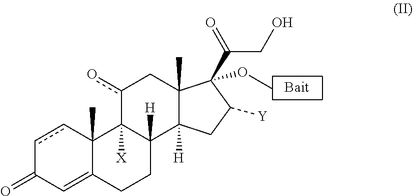
and pharmaceutically acceptable salts thereof, wherein: [symbol]
is an optional bond;
R is selected from the group consisting of H, C₁₋₄ alkyl, C₂₋₄ alkenyl, C₂₋₄ vinyl, and C₃₋₄ allyl;
X is selected from the group consisting of hallo, and H; and
Y is selected from the group consisting of CH₂ O, and NH.

8. The compound of claim 7, comprising the structure of Formula II, selected from the group consisting of 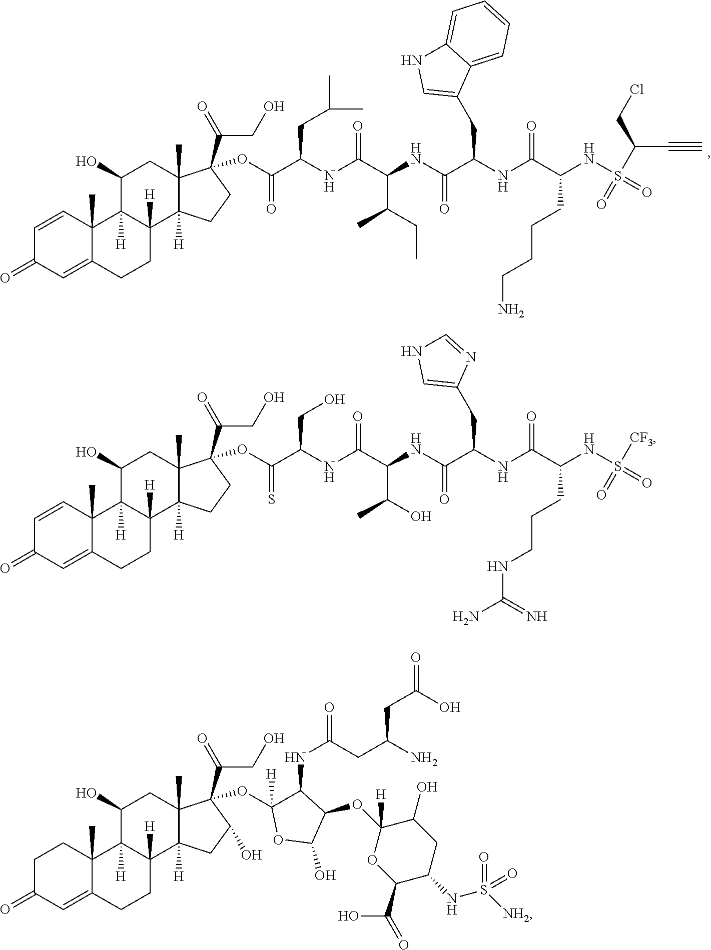
and pharmaceutically acceptable salts thereof.

9. A compound of claim 2 comprising a structure of Formula III: 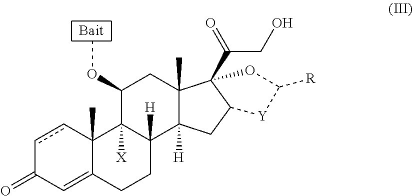
and pharmaceutically acceptable salt thereof, wherein: [symbol]
is an optional bond;
R is selected from the group consisting of H, C₁₋₄ alkyl, C₂₋₄ alkenyl, C₂₋₄ vinyl, and C₃₋₄ allyl;
X is selected from the group consisting of hallo, and H; and
Y is selected from the group consisting of CH₂ O, and NH.

10. The compound of claim 9, comprising the structure of Formula II, selected from the group consisting of 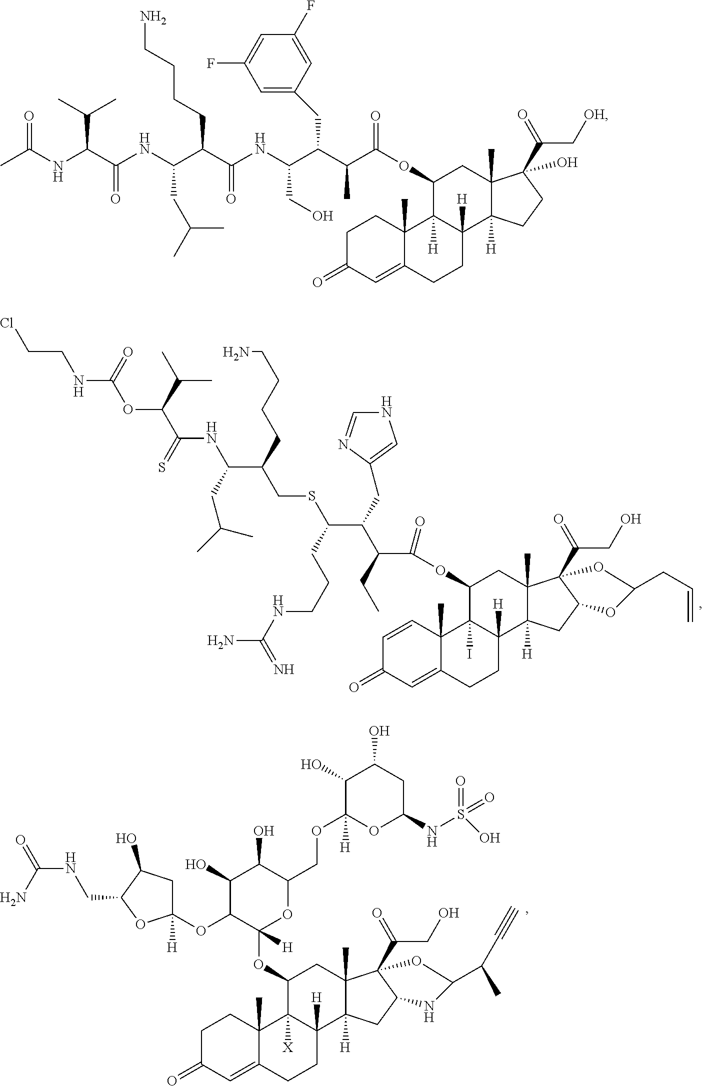
and pharmaceutically acceptable salts thereof.

11. A compound of claim 2, comprising a structure of Formula IV: 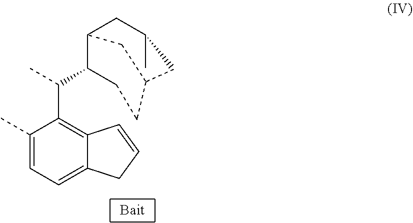
and pharmaceutically acceptable salts thereof, wherein: [symbol]
is an optional bond;
W at each instance is individually selected from the group consisting of Nitrile (CN), Sulfonamide (SO₂NHR for R═H, C₁₋₄ alkyl, C₂₋₄ alkenyl, C₂₋₄ vinyl, and C₃₋₄ allyl), or amide (CONR for SO₂NHR for R═H, C₁₋₄ alkyl, C₂₋₄ alkenyl, C₂₋₄ vinyl, and C₃₋₄ allyl) for each instance; and
Y is selected from the group consisting of C, and N.

12. The compound of claim 11, comprising the structure of Formula IV selected from the group consisting of 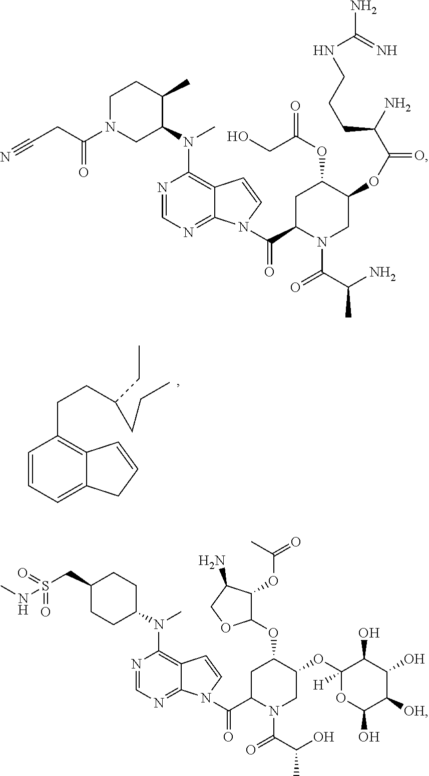
and pharmaceutically acceptable salt thereof.

13. A compound of claim 11, comprising a structure of Formula V: 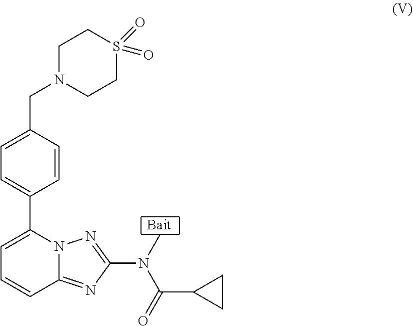 [SMILES: CN(C(=O)C1CC1)c1nc2cccc(-c3ccc(CN4CCS(=O)(=O)CC4)cc3)n2n1]
and pharmaceutically acceptable salts thereof.

14. A compound of claim 13, comprising the structure of Formula V selected from the group consisting of 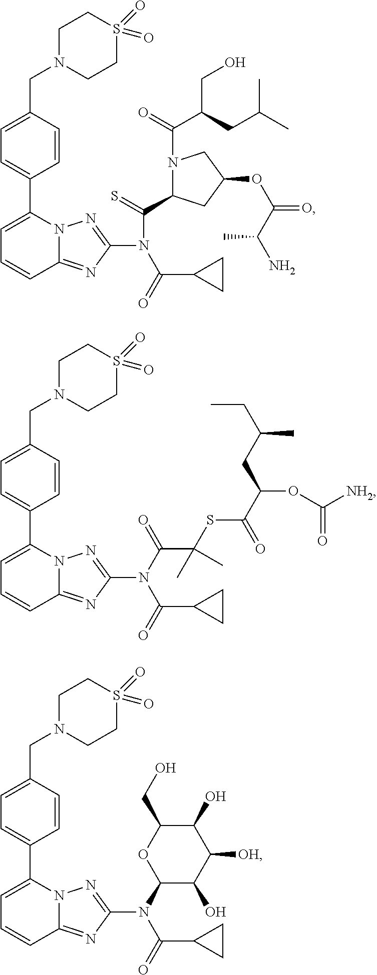
and pharmaceutically acceptable salts thereof.

15. A compound of claim 2, comprising a structure of Formula VI: 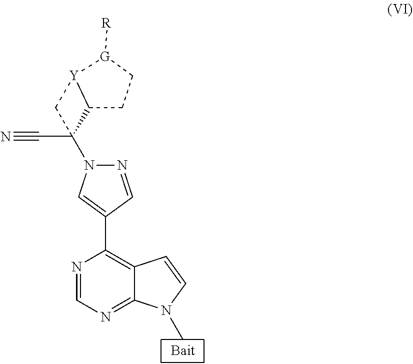
and pharmaceutically acceptable salts thereof, wherein: [symbol]
is an optional bond;
R is selected from the group consisting of H, C₁₋₄ alkyl, C₂₋₄ alkenyl, C₂₋₄ vinyl, and C₃₋₄ allyl;
G is selected from the group consisting of CH, —CO—, —SO—, and —SO₂—; and
Y is selected from the group consisting of CH, and N.

16. A compound of claim 15, comprising the structure of Formula VI, selected from the group consisting of 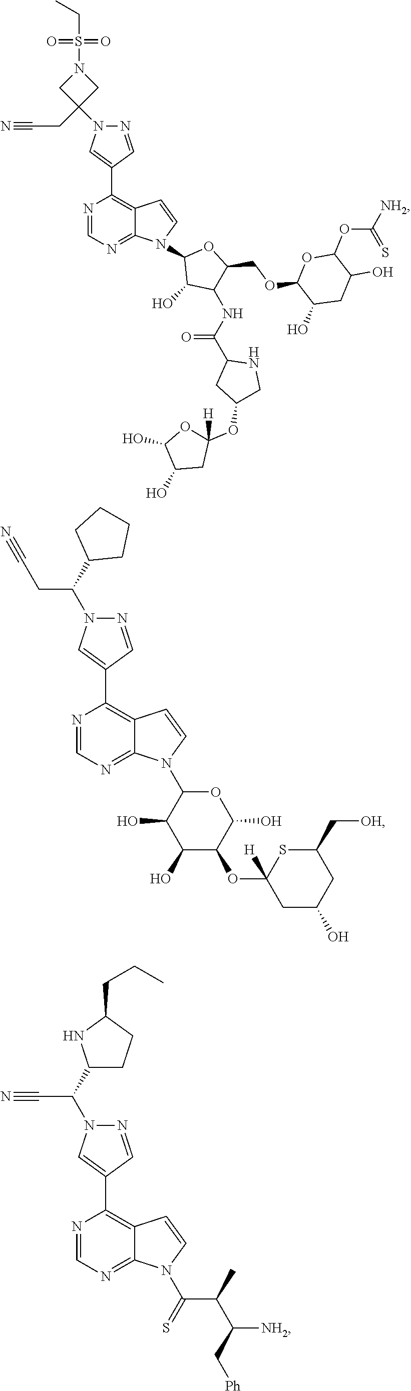
and pharmaceutically acceptable salts thereof.

17. A compound of claim 2, comprising a structure of Formula VI: 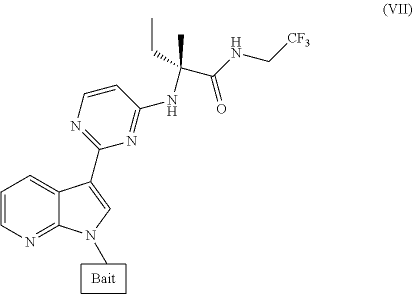
and pharmaceutically acceptable salts thereof.

18. A compound of claim 17, comprising the structure of Formula VII selected from the group consisting of 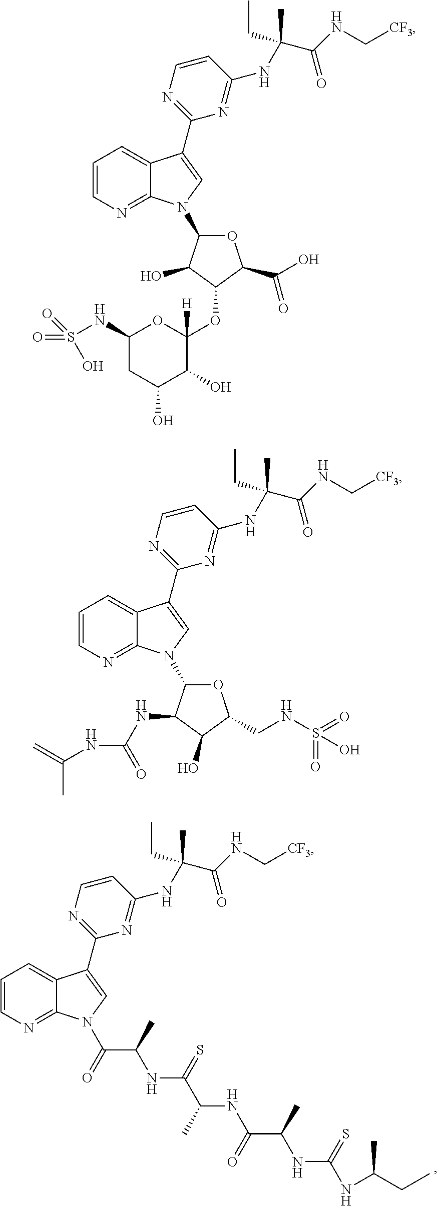 [SMILES: CC[C@@](C)(Nc1ccnc(-c2cn([C@H]3O[C@@H](C(=O)O)[C@H](O[C@H]4O[C@H](NS(=O)(=O)O)C[C@@H](O)[C@H]4O)[C@H]3O)c3ncccc23)n1)C(=O)NCC(F)(F)F ; C=C(C)NC(=O)N[C@@H]1[C@H](O)[C@@H](CNS(=O)(=O)O)O[C@H]1n1cc(-c2nccc(N[C@](C)(CC)C(=O)NCC(F)(F)F)n2)c2cccnc21 ; CC[C@H](C)NC(=S)N[C@H](C)C(=O)N[C@H](C)C(=S)N[C@H](C)C(=O)n1cc(-c2nccc(N[C@](C)(CC)C(=O)NCC(F)(F)F)n2)c2cccnc21]
and pharmaceutically acceptable salts thereof.

19. A compound of claim 2, comprising a structure of Formula VIII: 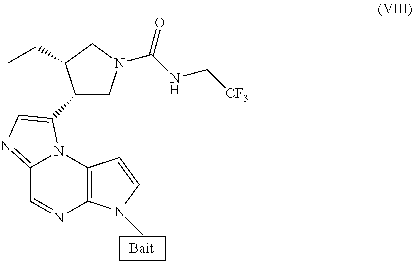 [SMILES: CC[C@@H]1CN(C(=O)NCC(F)(F)F)C[C@@H]1c1cnc2cnc3c(ccn3C)n12]
and pharmaceutically acceptable salts thereof.

20. A compound of claim 19, comprising the structure of Formula VII, selected from the group consisting of 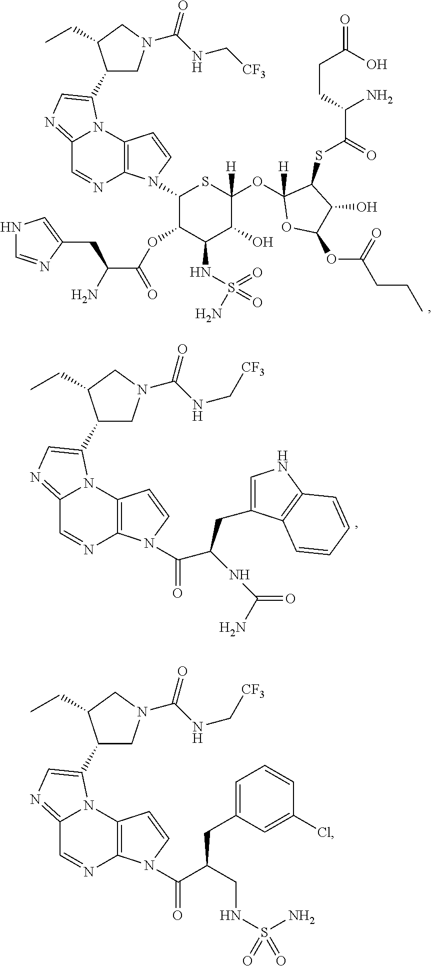
and pharmaceutically acceptable salt thereof.

21. A compound of claim 2, comprising a structure of Formula IX: 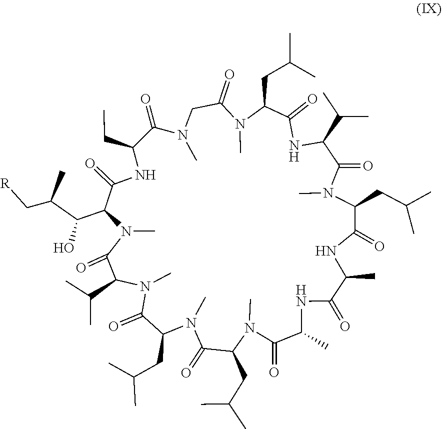
and pharmaceutically acceptable salts thereof, wherein
R is selected from the group consisting of C₁₋₈ alkyl, C₂₋₄ alkenyl, C₂₋₄ vinyl, and C₃₋₈ allyl.

22. The compound of claim 21, comprising the structure of Formula IX, selected from the group consisting of 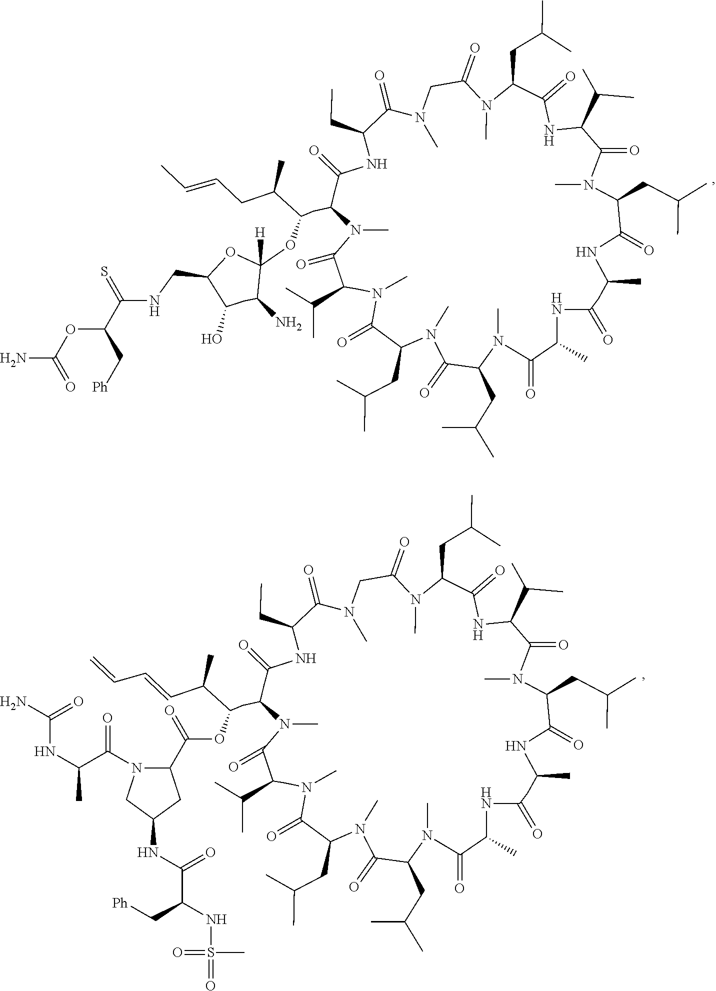 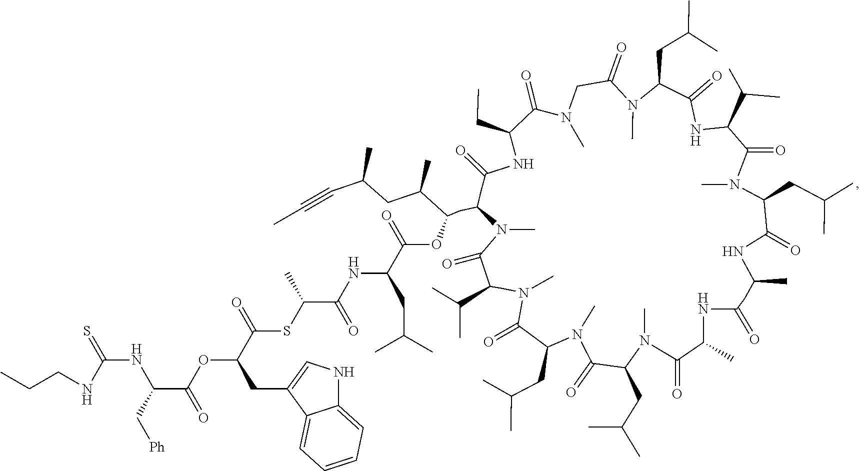 [SMILES: CC#C[C@@H](C)C[C@@H](C)[C@H](C[C@H]1CC(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)CC(=O)N[C@H](C)C(=O)N(C)[C@@H](CC(C)C)C(=O)N(C)[C@@H](CC(C)C)C(=O)N(C)C[C@@H](C(C)C)C(=O)N1C)OC(=O)[C@@H](CC(C)C)NC(=O)[C@@H](C)SC(=O)[C@@H](Cc1c[nH]c2ccccc12)OC(=O)[C@H](Cc1ccccc1)NC(=S)NCCC]
and pharmaceutically acceptable salts thereof.

23. A compound of claim 2, comprising the structure of Formula X: 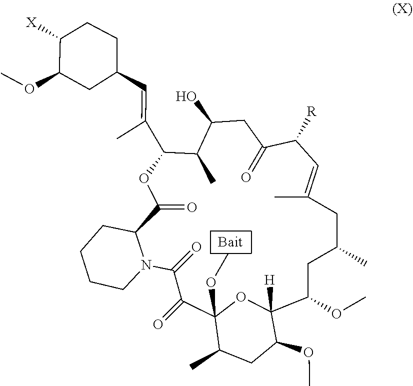
and pharmaceutically acceptable salts thereof, wherein:
R is selected from the group consisting of H, C₁₋₆ alkyl, C₂₋₆ alkenyl, C₂₋₆ vinyl, and C₃₋₆ allyl; and
X is selected from the group consisting of hallo, and OH.

24. The compound of claim 23, comprising the structure of Formula X, selected from the group consisting of 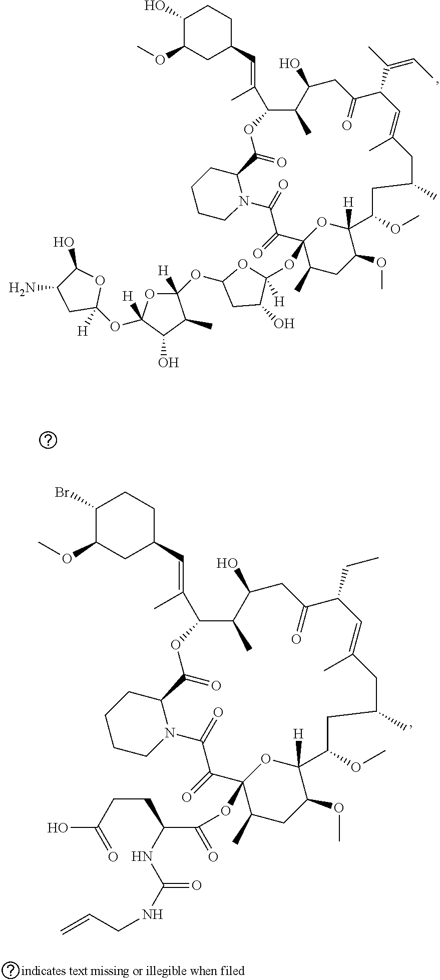
and pharmaceutically acceptable salts thereof.

25. A compound of claim 2, comprising a structure of Formula XI: 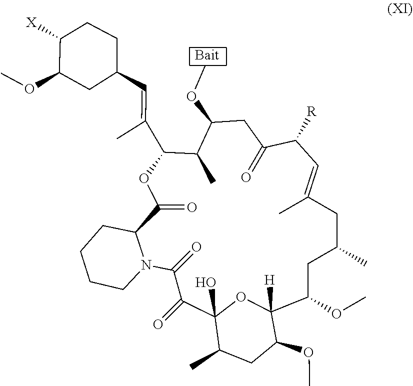
and pharmaceutically acceptable salts thereof, wherein:
R is selected from the group consisting of H, C₁₋₆ alkyl, C₂₋₆ alkenyl, C₂₋₆ vinyl, and C₃₋₆ allyl; and
X is selected from the group consisting of hallo, and OH;

26. A compound of claim 25, comprising the structure of Formula XI selected from the group consisting of 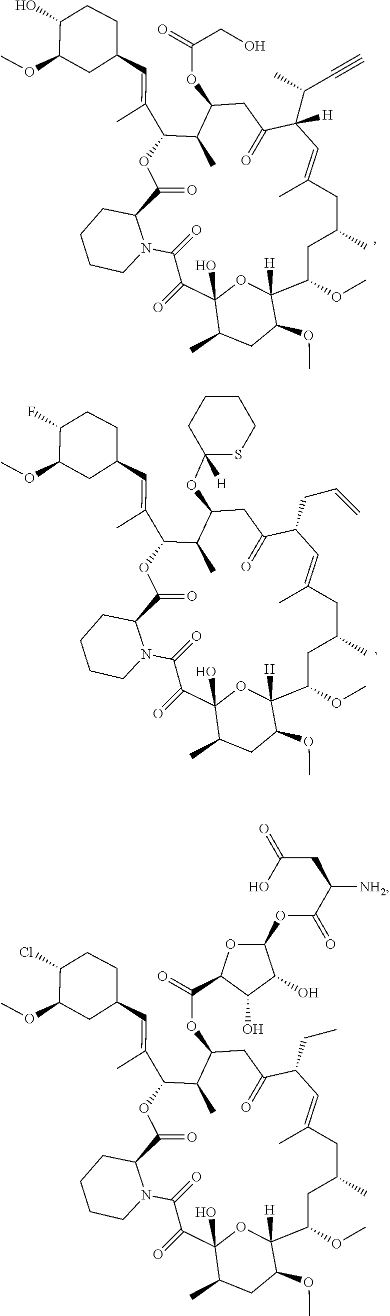
and pharmaceutically acceptable salts thereof.

27. A compound of claim 2, comprising a structure of Formula XII: 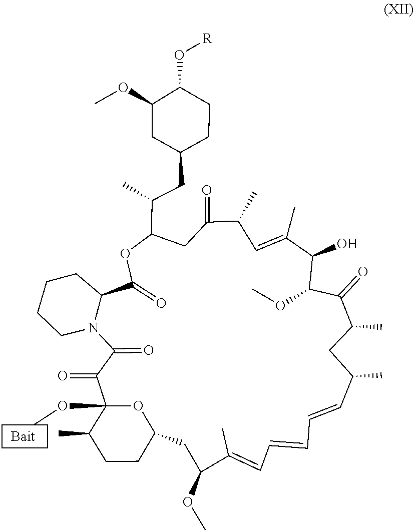
and pharmaceutically acceptable salts thereof, wherein
R is selected from the group consisting of either H, or one of the following groups with an OH substituent: C₁₋₈ alkyl, C₂₋₈ alkenyl, C₂₋₈ vinyl, and C₃₋₈ allyl.

28. A compound of claim 27, comprising the structure of Formula XII, selected from the group consisting of 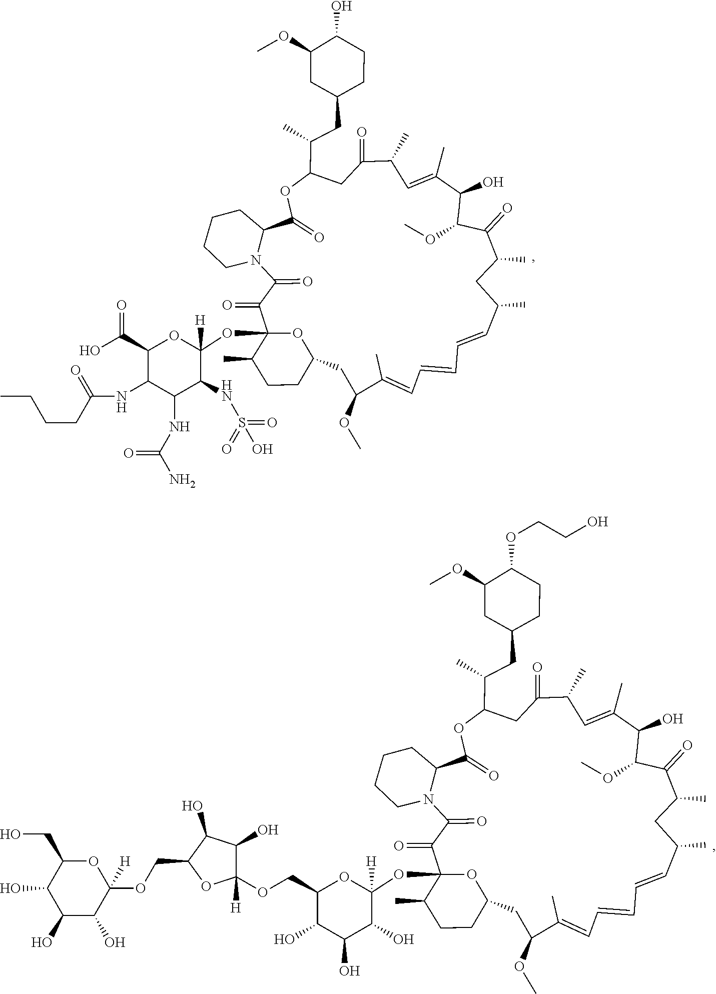 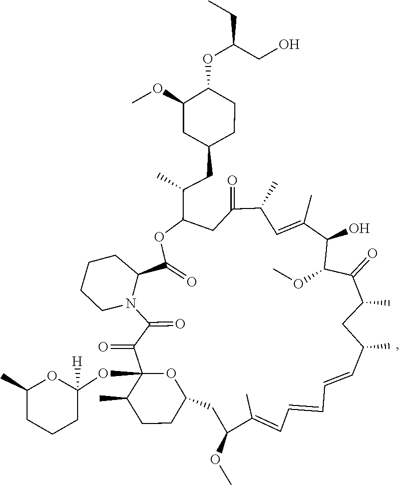
and pharmaceutically acceptable salts thereof

29. A compound of claim 2, comprising a structure of Formula XIII: 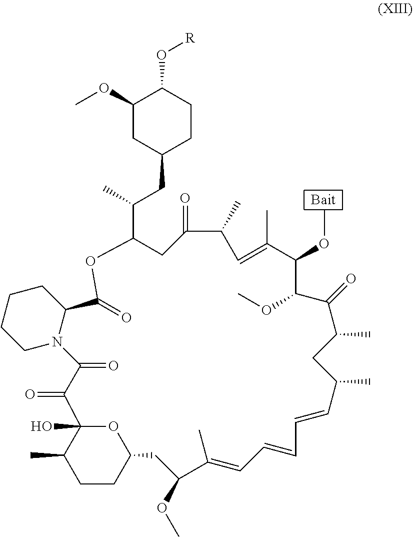
and pharmaceutically acceptable salts thereof, wherein
R is selected from the group consisting of either H, or one of the following groups with an OH substituent: C₁₋₈ alkyl, C₂₋₈ alkenyl, C₂₋₈ vinyl, and C₃₋₈ allyl.

30. A compound of claim 29, comprising the structure of Formulas XIII, selected from the group consisting of 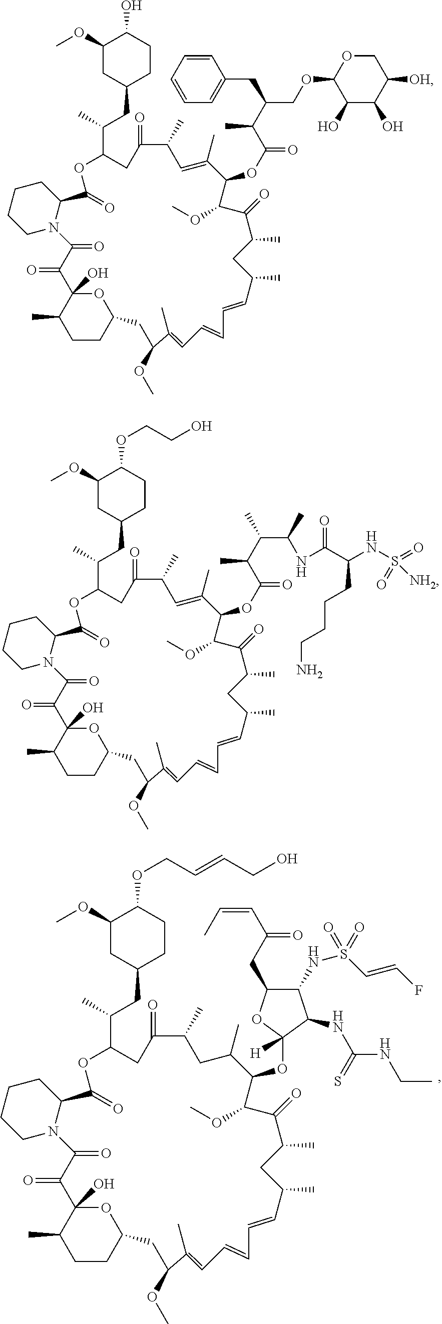
and pharmaceutically acceptable salts thereof.

31. A compound of claim 2, comprising a structure of Formula XIV: 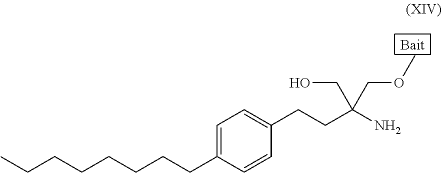 [SMILES: CCCCCCCCc1ccc(CCC(N)(CO)COC)cc1]
and pharmaceutically acceptable salts thereof.

32. A compound of claim 31 having the structure of Formula XIV, selected from the group consisting of 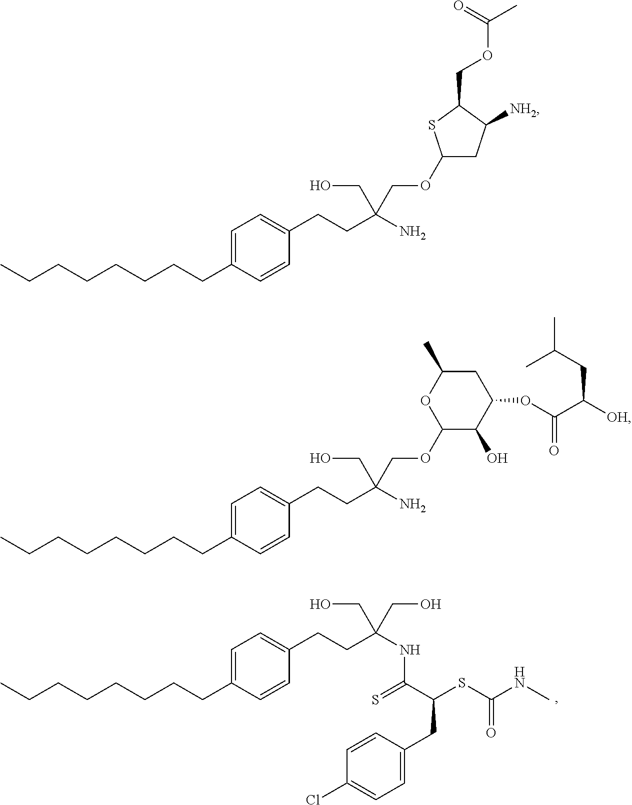
and pharmaceutically acceptable salts thereof.

33. A compound of claim 2, comprising a structure of Formula XV: 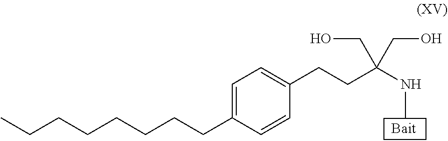 [SMILES: CCCCCCCCc1ccc(CCC(CO)(CO)NC)cc1]
and pharmaceutically acceptable salts thereof.

34. A compound of claim 33, comprising the structure of Formula XV, selected from the group consisting of 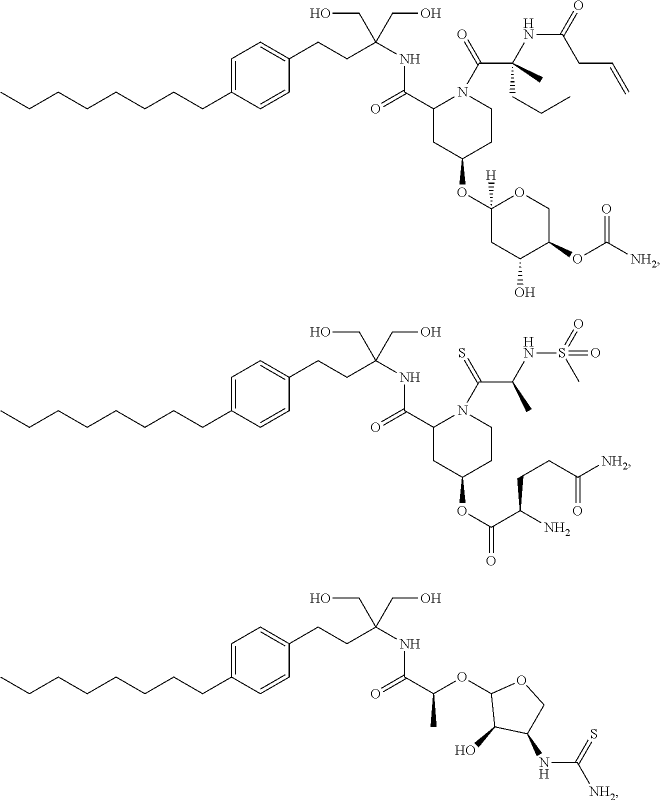 [SMILES: C=CCC(=O)N[C@](C)(CCC)C(=O)N1CC[C@@H](O[C@@H]2C[C@@H](O)[C@H](OC(N)=O)CO2)CC1C(=O)NC(CO)(CO)CCc1ccc(CCCCCCCC)cc1 ; CCCCCCCCc1ccc(CCC(CO)(CO)NC(=O)C2C[C@H](OC(=O)[C@H](N)CCC(N)=O)CCN2C(=S)[C@H](C)NS(C)(=O)=O)cc1 ; CCCCCCCCc1ccc(CCC(CO)(CO)NC(=O)[C@H](C)OC2OC[C@@H](NC(N)=S)[C@H]2O)cc1]
and pharmaceutically acceptable salts thereof.

35. A compound of claim 2, comprising a structure of Formula XVI: 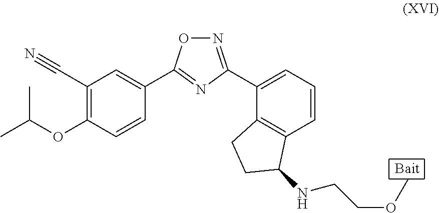 [SMILES: COCCN[C@H]1CCc2c(-c3noc(-c4ccc(OC(C)C)c(C#N)c4)n3)cccc21]
and pharmaceutically acceptable salts thereof.

36. A compound of claim 35, comprising the structure of Formula XVI, selected from the group consisting of 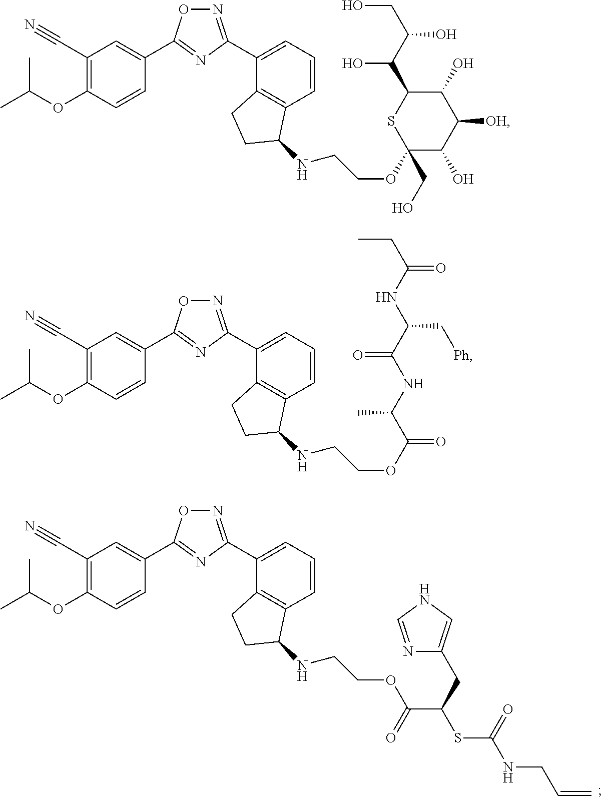
and pharmaceutically acceptable salts thereof.

37. A compound of claim 2, comprising a structure of Formula XVII: 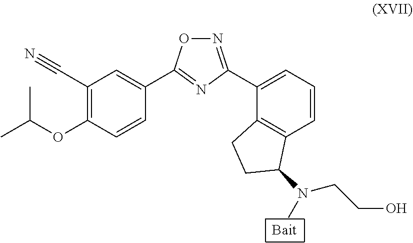 [SMILES: CC(C)Oc1ccc(-c2nc(-c3cccc4c3CC[C@@H]4N(C)CCO)no2)cc1C#N]
and pharmaceutically acceptable salts thereof.

38. A compound if claim 37, comprising the structure of Formula XVIII, selected from the group consisting of 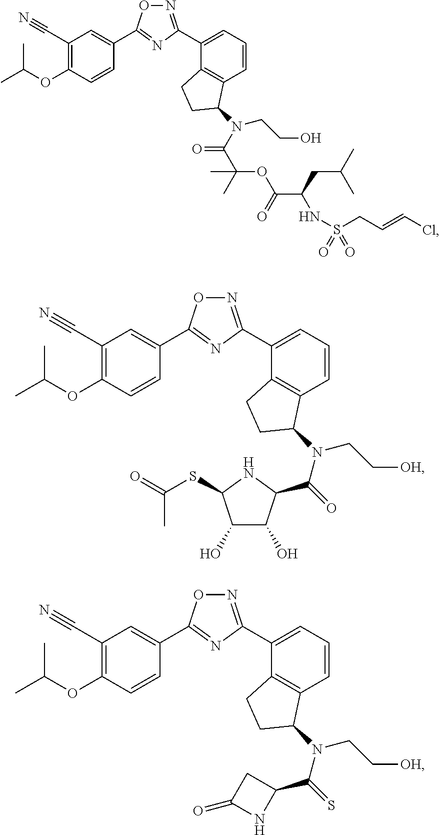
and pharmaceutically acceptable salts thereof.

39. A compound of claim 2, comprising a structure of Formula XVIII: 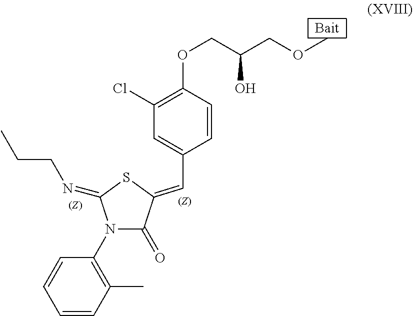 [SMILES: CCC/N=C1\S/C(=C\c2ccc(OC[C@H](O)COC)c(Cl)c2)C(=O)N1c1ccccc1C]
and pharmaceutically acceptable salts thereof.

40. A compound of claim 39, comprising the structure of Formula XVIII, selected from the group consisting of 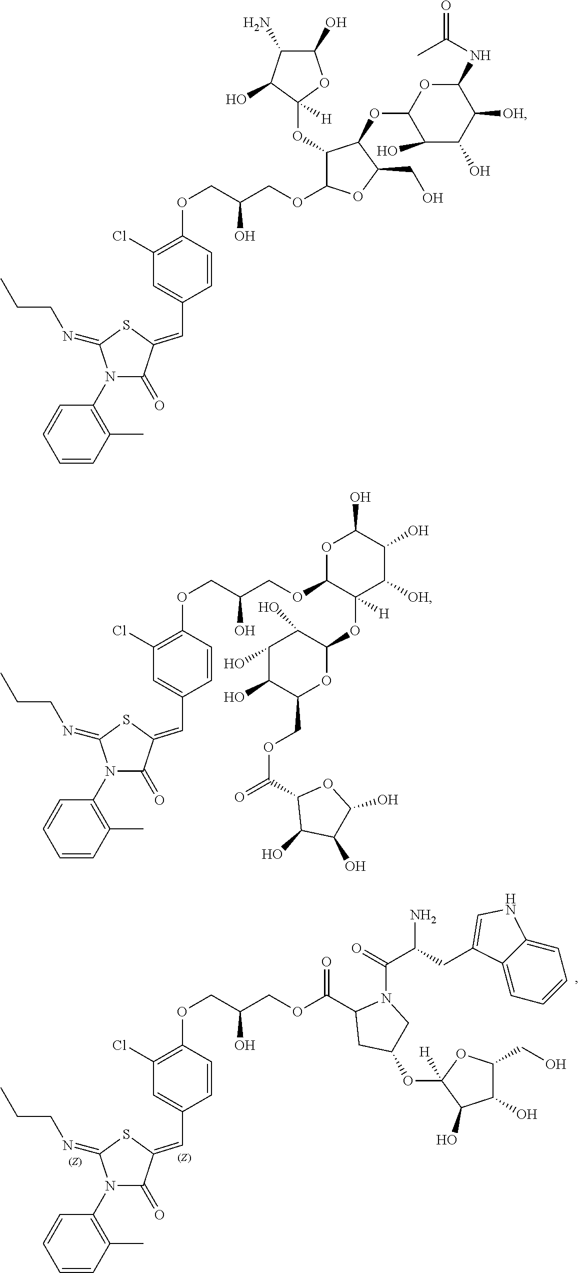 [SMILES: CCC/N=C1\S/C(=C\c2ccc(OC[C@H](O)COC3O[C@H](CO)[C@H](OC4O[C@@H](NC(C)=O)[C@@H](O)[C@H](O)[C@H]4O)[C@H]3O[C@@H]3O[C@H](O)[C@@H](N)[C@@H]3O)c(Cl)c2)C(=O)N1c1ccccc1C ; CCC/N=C1\S/C(=C\c2ccc(OC[C@H](O)CO[C@H]3O[C@@H](O)[C@H](O)[C@H](O)[C@H]3O[C@H]3O[C@@H](COC(=O)[C@@H]4O[C@H](O)[C@@H](O)[C@H]4O)[C@@H](O)[C@H](O)[C@@H]3O)c(Cl)c2)C(=O)N1c1ccccc1C ; CCC/N=C1\S/C(=C\c2ccc(OC[C@H](O)COC(=O)C3C[C@@H](O[C@H]4O[C@H](CO)[C@H](O)[C@H]4O)CN3C(=O)[C@H](N)Cc3c[nH]c4ccccc34)c(Cl)c2)C(=O)N1c1ccccc1C]
and pharmaceutically acceptable salts thereof.

41. A compound of claim 2, comprising a structure of Formula XIX: 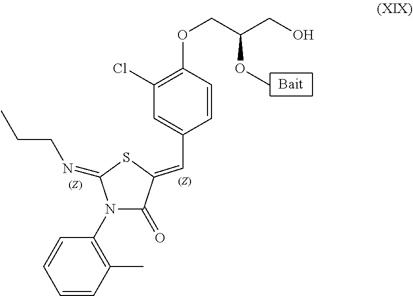 [SMILES: CCC/N=C1\S/C(=C\c2ccc(OC[C@@H](CO)OC)c(Cl)c2)C(=O)N1c1ccccc1C]
and pharmaceutically acceptable salts thereof.

42. A compound of claim 41, comprising the structure of Formula XIX, selected from the group consisting of 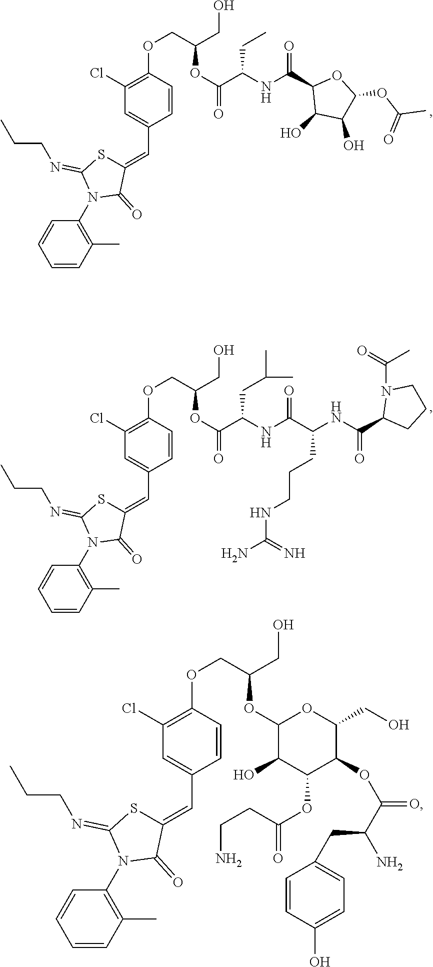
and pharmaceutically acceptable salts thereof.

43. A pharmaceutical formulation comprising a CSIM compound of any one of claims 1-42, and a pharmaceutically acceptable carrier.

44. The pharmaceutical formulation of claim 43, further comprising an immunomodulatory or anti-proliferative compound different than the CSIM compound.

45. A method of treating a proliferative disorder in a subject, comprising administering to the subject an amount of the pharmaceutical formulation of any one of claims 43 and 44 effective to inhibit or reduce a symptom of the proliferative disorder.

46. A method of inhibiting the ability of an immune cell to proliferate, comprising contacting the cell with an amount of any one of claims 1-42 effective to inhibit the cell's ability to proliferate.

47. A method of synthesizing a compound of claim 2, comprising the protocols set forth in FIGS. 2-4.

48. A method of synthesizing a CSIM having structure: 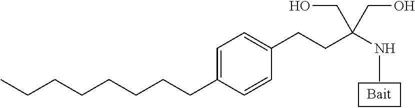 [SMILES: CCCCCCCCc1ccc(CCC(CO)(CO)NC)cc1]
comprising the protocol set forth in FIG. 5.
